SEQUENCE LISTING (1 sequence)

SEQ ID NO: 1 - DNA, 153 bp, Homo sapiens
gccgggcgcggtggcgcgtgcctgtagtcccagctactcgggaggctgaggctggaggatcgcttgagtccaggagttctgggctgtagtgcgctatgccgatcggaatagccactgcactccagcctgggcaacatagcgagaccccgtctc

(19) United States
(12) Patent Application Publication
(10) Pub. No.: US 2022/0145359 A1
(43) Pub. Date: May 12, 2022

(54) METHODS FOR TARGETED DEPLETION OF NUCLEIC ACIDS

(71) Applicant: JUMPCODE GENOMICS, INC., San Diego, CA (US)

(72) Inventor: Keith BROWN, Carlsbad, CA (US)

(21) Appl. No.: 17/430,102

(22) PCT Filed: Feb. 11, 2020
(86) PCT No.: PCT/US2020/017707

§ 371 Date: Aug. 11, 2021

Related U.S. Application Data
(60) Provisional application No. 62/804,587, filed on Feb. 12, 2019.

(51) Int. Cl. C12Q 1/6806; C12N 9/22; C12N 15/11; C12N 15/10

(52) U.S. Cl. CPC C12Q 1/6806; C12N 9/22; C12N 2310/20; C12N 15/1041; C12N 2800/80; C12N 15/11

(57) ABSTRACT

Disclosed herein are compositions and methods related to the elimination of a first nucleic acid and enrichment of a second nucleic acid in a sample, for example to exclude the first nucleic acid from downstream analysis or sequencing, or to exclude such sequences from a downstream data set.

### CROSS-REFERENCE

This application claims the benefit of U.S. Provisional Application No. 62/804,587, filed Feb. 12, 2019, which application is incorporated herein by reference in its entirety.

### BACKGROUND

The disclosure herein relates to the field of molecular biology, such as methods and compositions for depleting a target nucleic acid from a sample, enriching for sequences of interest from a sample, and/or partitioning of sequences from a sample. The methods and compositions are applicable to biological, clinical, forensic, and environmental samples.

Many human clinical DNA samples or extracted DNA samples taken from tissue, fluids, or other host material samples contain highly abundant nucleic acids having sequences that have little informative value and increase the cost of sequencing. Some methods such as differential lysis of cell types have been developed to address this issue, but these methods are often time-consuming and can be inefficient. Therefore, there is a need for developing a more viable method for depleting the target nucleic acid in a sample.

### SUMMARY

Provided herein are methods, compositions, and systems that can selectively deplete target nucleic acids from a sample, enriching nucleic acids of interest from a sample, and/or partitioning of nucleic acids from a sample. The methods and compositions are applicable to biological, clinical, forensic, and environmental samples. Methods of depleting a first nucleic acid from a sample can include one or more of the steps of providing a sample comprising the first nucleic acid and a second nucleic acid; capping 5′ and 3′ ends of the first nucleic acid and the second nucleic acid, such as using a cap that is resistant to exonuclease activity; contacting the sample to a moiety having endonuclease activity to form at least one cleaved first nucleic acid, wherein the endonuclease cleaves the first nucleic acid but does not cleave the second nucleic acid; and contacting the sample to an exonuclease. Some examples of the nucleic acids to be depleted in the sample can be host nucleic acids, repetitive regions within a sample such as transposon regions, Alu repeats, ribosomal DNA, high copy mitochondrial DNA, or other nucleic acids present in high copy number or conveying low information content sequence. Other examples are consistent with the disclosure herein, such that any high copy, redundant or otherwise undesired nucleic acid is selectively removed from a sample. Some of examples of the nucleic acid of interest include but are not limited to pathogen or other non-host nucleic acids within a host sample, tumor nucleic acids or other rare mutant nucleic acids in a non-mutant background, fetal DNA in a maternal sample, naturally occurring stable alleles, and alleles arising during the life of a subject. Other examples are consistent with the disclosure herein, such that any low copy, rare or otherwise desired nucleic acid is enriched through selective depletion of other nucleic acids in a sample or library.

Provided herein, in certain aspects, are methods of depleting a first nucleic acid from a sample. In some cases, methods herein comprise providing a sample comprising the first nucleic acid and a second nucleic acid; capping 5′ and 3′ ends of the first nucleic acid and the second nucleic acid; and contacting the sample to an endonuclease to form at least one cleaved first nucleic acid, wherein the endonuclease cleaves the first nucleic acid but does not cleave the second nucleic acid. In some cases, methods herein comprise contacting the sample to an exonuclease. In some cases, capping comprises modifying the 5′ or 3′ ends of the first and second nucleic acids to make the first and the second nucleic acids resistant to exonuclease degradation. In some cases, capping comprises attaching adaptors to the 5′ and 3′ ends of the first nucleic acid and the second nucleic acid. In some cases, the adaptor is a hairpin or a linear adaptor. In some cases, the linear adaptor is selected from the group consisting of phosphorthioate, 2-O methyl, inverted dT, inverted ddT, phosphorylation, and C3 spacers. In some cases, the endonuclease is a restriction enzyme specific to at least one site on the first nucleic acid. In some cases, the endonuclease comprises at least one selected from Clustered Regulatory Interspaced Short Palindromic Repeat (CRISPR)/Cas system protein-guide RNA (gRNA) complexes, Zinc Finger Nucleases (ZFN), and Transcription activator like effector nucleases. In some cases, the gRNAs are complementary to at least one site on the first nucleic acid to generate cleaved first nucleic acids capped only on one end. In some cases, the endonuclease comprises an Alu specific restriction enzyme. In some cases, the first nucleic acid comprises at least one sequence that maps to at least one nucleic acid selected from the group consisting of AluI, AsuHPI, Bpu10I, BssECI, BstDEI, BstMAI, HinfI, and BstTUI. In some cases, the cleaved first nucleic acid is capped at only one end. In some cases, the cleaved first nucleic acid has a first end that is attached to an adaptor and a second end that is not attached to an adaptor. In some cases, the method comprises extracting the first and second nucleic acids from the sample and purifying the first and second nucleic acids. In some cases, the first and second nucleic acids comprise any one of single stranded DNA, double stranded DNA, single stranded RNA, double stranded RNA, cDNA, synthetic DNA, artificial DNA, and DNA/RNA hybrids. In some cases, the method comprises amplifying the second nucleic acid. In some cases, the method comprises sequencing the second nucleic acid. In some cases, the method comprises sequencing the second nucleic acid through a second-generation sequencing method. In some cases, the method comprises sequencing the second nucleic acid through a nanopore sequencing method. In some cases, the first nucleic acid comprises a nucleic acid from a human. In some cases, the first nucleic acid comprises a host nucleic acid. In some cases, the first nucleic acid comprises a repetitive nucleic acid. In some cases, the first nucleic acid comprises a centromere nucleic acid. In some cases, the first nucleic acid comprises a transposon. In some cases, the first nucleic acid comprises an Alu element. In some cases, the second nucleic acid comprises a microbiome nucleic acid. In some cases, the second nucleic acid comprises an oncogenic nucleic acid. In some cases, the second nucleic acid comprises a symbiont nucleic acid. In some cases, the second nucleic acid comprises a single-copy region of a haploid genome. In some cases, the second nucleic acid comprises a nucleic acid from a pathogen. In some cases, the pathogen is selected from the group consisting of a virus, a bacterium, a fungus, and a protozoon. In some cases, the method comprises sequencing the second nucleic acid and determining the type of the pathogen. In some cases, the second nucleic acid comprises a nucleic acid from a tumor. In some cases, the sample wherein the sample is selected from saliva, blood, plasma, serum, mucous, feces, urine, cerebrospinal fluid (CSF), skin, tissue, and bone.

Further provided herein, in certain aspects, are compositions comprising a mixture of a first nucleic acid and a second nucleic acid, wherein the first nucleic acid and the second nucleic acid are capped at 3′ and 5′ ends, and wherein the first nucleic acid is complexed to an endonuclease and the second nucleic acid is not complexed to the endonuclease. In some cases, the endonuclease comprises at least one selected from Clustered Regulatory Interspaced Short Palindromic Repeat (CRISPR)/Cas system protein-gRNA complex, Zinc Finger Nucleases (ZFN), and Transcription activator like effector nucleases. In some cases, the endonuclease comprises a Clustered Regulatory Interspaced Short Palindromic Repeat (CRISPR)/Cas system protein-guide RNA (gRNA) complexes. In some cases, the gRNA is complementary to at least one site on the first nucleic acid to generate cleaved first nucleic acids capped only on one end. In some cases, the endonuclease comprises an Alu specific restriction enzyme. In some cases, the first nucleic acid comprises at least one sequence that maps to at least one nucleic acid selected from the group consisting of AluI, AsuHPI, Bpu10I, BssECI, BstDEI, BstMAI, HinfI, and BstTUI. In some cases, the first nucleic acid comprises a repetitive region. In some cases, the first nucleic acid comprises an Alu repeat. In some cases, the first nucleic acid comprises a nucleic acid from a human. In some cases, the second nucleic acid comprises a nucleic acid from a pathogen. In some cases, the pathogen is selected from the group consisting of a virus, bacterial, fungus, and protozoa. In some cases, the second nucleic acid comprises a nucleic acid from a tumor. In some cases, the first nucleic acid comprises a host nucleic acid. In some cases, the first nucleic acid comprises a repetitive nucleic acid. In some cases, the first nucleic acid comprises a centromere nucleic acid. In some cases, the second nucleic acid comprises a microbiome nucleic acid. In some cases, the second nucleic acid comprises an oncogenic nucleic acid. In some cases, the second nucleic acid comprises a symbiont nucleic acid. In some cases, the second nucleic acid comprises a single-copy region of a haploid genome. In some cases, the first nucleic acid comprises a transposon. In some cases, the first nucleic acid comprises an Alu element.

### BRIEF DESCRIPTION OF THE DRAWINGS

Some understanding of the features and advantages of the present invention will be obtained by reference to the following detailed description that sets forth illustrative embodiments, in which the principles of the invention are utilized, and the accompanying drawings.

FIG. 1 depicts a work flow of an exemplified depletion of a first nucleic acid and enrichment of a second nucleic acid.

FIG. 2 depicts a map of Alu sequences in the human genome.

### DETAILED DESCRIPTION

Disclosed herein are methods, systems, and compositions for depleting a first nucleic acid and enriching a second nucleic acid in a sample comprising both the first and the second nucleic acids. The first nucleic acid is a host nucleic acid and the method described herein relates to a method for host depletion. Methods involve one or more of the following steps, performed independently or in combination: a) protection of the first and second nucleic acid molecules in the sample, rendering them immune to degradation via exonuclease; b) endonuclease digestion of sequence motifs found only in the first nucleic acid (e.g. host genome); c) these new “unprotected” ends of molecules are then exposed to exonuclease digestion, thereby degrading the first nucleic acid (e.g., host DNA). The remaining intact second nucleic acids (e.g., non-host nucleic acid) go through standard library preparation. The resulting library can be sequenced, and when the first nucleic acid is from a pathogen, the pathogen can be identified. This allows for both novel and known pathogens to be detected in a single workflow.

Through practice of the disclosure herein, one can selectively enrich nucleic acids of interest, or selectively deplete nucleic acids that are not of interest from a sample, and thus more accurately and efficiently detect pathogen, tumor, fetal DNA, alleles, and other nucleic acids of interest in a sample.

Viewing pathogen detection as an example, whole genome sequencing, or shot gun sequencing, offers a promising solution to detect pathogens. A challenge can be that many sample types contain an abundance of host molecules, limiting the sensitivity of shot gun sequencing to detect non-host pathogen nucleic acids and increasing the amount of sequence that must be generated so as to obtain reads representative of rare molecules in the sample, such as molecules derived from a pathogen or other exogenous organism on a host derived nucleic acid sample. A similar challenge presents itself in the identification of any rare or single copy nucleic acid in a sample that also comprises high copy or non-interest nucleic acids. Pathogen detection can be used in a number of applications including, but not limited to, an infectious disease outbreak, detecting a pathogen in an immune compromised individual, detecting pathogens in a blood bank, detection of pathogens in veterinary or agricultural samples, detection of plant pathogens in agricultural samples, removal of bacterial contaminant from saliva samples, mitochondrial nucleic acid depletion, or chloroplast nucleic acid depletion.

A number of sample preparation approaches have been proposed to address these challenges. Differential lysis of cell types has been described. For example, human cells are lysed via one lysis method, DNA from those cells are degraded via exonuclease, then the remaining non-human cells are lysed and prepared for sequencing. Another method aims to degrade methylated DNA, more abundant in human DNA than pathogen DNA, has also been described. These approaches are specific to a particular cell type or nucleic acid modifications.

Alternative approaches such as genome fractioning have been described. These methods use a pool of CRISPR guide RNAs to digest host DNA/cDNA molecules after library generation. This approach is simple, fast and cost effective. However, a large number of guides are required to direct CRISPR endonucleases to make a double stranded cut in particular sequencing libraries so as to render them incapable of amplification via universal adapter sequences.

Provided herein are compositions and methods for selective target enrichment or selective background depletion that are readily performed on a broad range of samples and that do not require amplification for depletion.

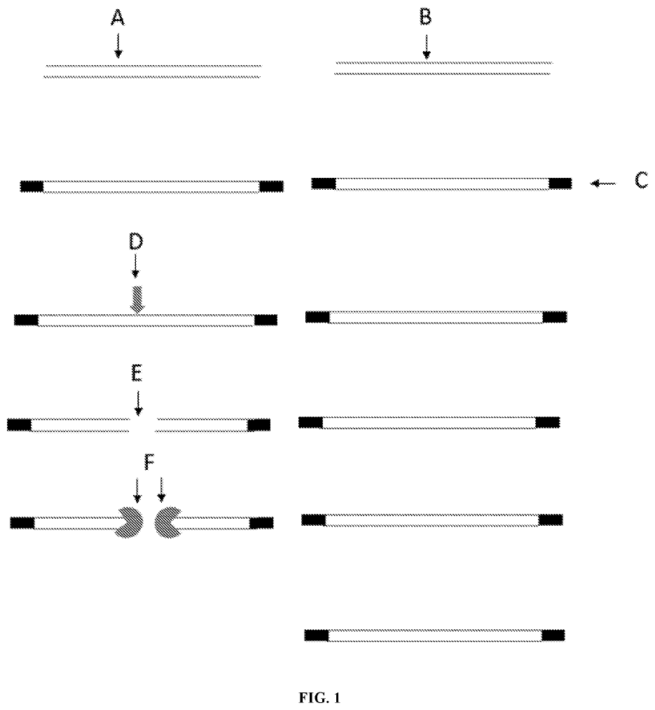

FIG. 1 shows an example of the steps for depleting a first nucleic acid (e.g., host nucleic acid) and enriching a second nucleic acid (e.g., non-host nucleic acid). Nucleic acid A represents the first nucleic acid (e.g., host DNA molecule, redundant sample nucleic acid or other redundant nucleic acid to be depleted), and nucleic acid B represents the second nucleic acid (e.g., pathogen DNA molecule, allele, cancer mutant nucleic acid, or high information segment of a genome). C shows an example of the nucleic acid end protection (ligation or tagmentation with hairpin or modified ends) that renders nucleic acids resistant for exonuclease degradation, and D shows the specific endonuclease recognition site (restriction enzyme (RE), CRISPR complementary site, or other site) of the first nucleic acid that facilitates targeted removal. E shows the exposed end of the cleaved first nucleic acids after endonuclease digestion, and F shows exonuclease digestion of the cleaved first nucleic acids. Only the second nucleic acid, or a population of nucleic acids sharing as a common trait the absence of the cleavage site (that is, a nucleic acid lacking the cleavage site such as non-host DNA, nonredundant DNA or high information segments of a genome as described herein) remains in the sample after the steps shown in FIG. 1. In some cases, the exonuclease is Exonuclease III or BAL-31, though a number of exonucleases are compatible with the disclosure herein.

Samples of various nucleic acid sources are compatible with the disclosure herein. Some samples are heterogeneous RNA/DNA compositions as starting materials. Accordingly, disclosed herein are methods of enriching or depleting certain nucleic acids from a total nucleic acid sample comprising RNA and DNA. RNA is first converted into double stranded cDNA. Both double stranded cDNA and genomic DNA molecules are protected by addition of end adapters to render them immune to exonuclease degradation. cDNA and DNA molecules are subjected to endonuclease digestion as described herein, so as to cleave a first nucleic acid (e.g., host nucleic acid, repetitive nucleic acid or other nucleic acid to be depleted) specific sequence motifs. Exposed ends of the unprotected endonuclease-digested first nucleic acid act as entry points for degradation of cleaved fragments via exonuclease digestion. The remaining uncleaved ‘second nucleic acid’ molecules are converted into sequencing libraries, sequenced and the data is analyzed to identify enriched nucleic acids such as pathogen or cancer nucleic acids, for example, present in the sample.

A number of sequence-specific cleavage approaches can be used to deplete target nucleic acids so as to enrich for nucleic acid of interest. These techniques, including Zinc Finger Nucleases (ZFN), Transcription activator like effector nucleases and Clustered Regulatory Interspaced Short Palindromic Repeat/Cas based RNA guided DNA nuclease (CRISPR/Cas9) allow for sequence specific degradation of double stranded DNA. Alternately, restriction endonuclease, particularly restriction endonucleases that have cleavage specificity that targets particular regions to be depleted while preferably leaving other nucleic acid molecules uncleaved, are also compatible with the disclosure herein. In some embodiments, a repeat-region specific endonuclease such as an Alu restriction endonuclease or other transposon or repeat region specific endonuclease is selected so as to deplete the corresponding nucleic acids from a sample. These techniques can be used to, for example, cleave the first nucleic acid at one or more sites to generate an exposed end or set of exposed ends available for exonuclease degradation. The ability to target sequence specific locations for double stranded DNA cuts makes these genome editing tools compatible with depletion of a redundant or otherwise undesired target nucleic acid in the sample.

A sample subjected to selective depletion comprises sequence of the first nucleic acid and the second nucleic acid. In some embodiments a target sample comprises non-repetitive sequence and repetitive sequence. In some embodiments a target sample comprises single-copy sequence and multi-copy sequence. In some cases a host sample is fragmented and differentially degraded so as, for example, to selectively remove repetitive regions of a genome while leaving high-information regions undegraded and therefore selectively enriched. In some embodiments, a sample comprises blood, serum, plasma, nasal swab or nasopharyngeal wash, saliva, urine, gastric fluid, spinal fluid, tears, stool, mucus, sweat, earwax, oil, glandular secretion, cerebral spinal fluid, tissue, semen, vaginal fluid, interstitial fluids, including interstitial fluids derived from tumor tissue, ocular fluids, spinal fluid, throat swab, breath, hair, finger nails, skin, biopsy, placental fluid, amniotic fluid, cord blood, emphatic fluids, cavity fluids, sputum, pus, microbiota, meconium, breast milk and/or other excretions. In some cases, a blood sample comprises circulating tumor cells or cell free DNA, such as tumor DNA or fetal DNA.

Provided herein are methods, compositions and kits related to the selective enrichment of nucleic acids of interest, such as selective enrichment of pathogen nucleic acids, symbiote nucleic acids, microbiome nucleic acids, high information regions, cancer alleles, or other nucleic acids of interest in a sample.

In some cases, the first nucleic acid is from a host. In some cases, the first nucleic acid is from one or more hosts selected from the group consisting of mammals, such as a human, cow, horse, sheep, pig, monkey, dog, cat, gerbil, bird, mouse, and rat, or any mammalian laboratory model for a disease, condition or other phenomenon involving rare nucleic acids. In some cases, the first nucleic acid is from a human. Some of examples of the second nucleic acid, e.g., the nucleic acid of interest can be from pathogens, microbiomes, tumor, fetal DNA in a maternal sample, alleles, and mutant alleles. In some cases, the second nucleic acid is from a non-host. In some cases, the second nucleic acid is from a prokaryotic organism. In some cases, the second nucleic acid is from one or more selected from the group consisting of a eukaryote, virus, bacterial, fungus, and protozoa. In some embodiments, the second nucleic acid can be from tumor cells. In some embodiments, the second nucleic acid can be fetal DNA in a maternal sample. In some embodiments, the second nucleic acid can be alleles or mutant alleles. Microbiomes are also sources of second nucleic acids consistent with the disclosure herein, as are other examples apparent to one of skill in the art.

In some cases, the first nucleic acid and the second nucleic acid are capped at the 5′ and 3′ ends in order to protect the ends from exonuclease digestion. In some embodiments, the first nucleic acid and the second nucleic acid are capped by attaching an adapter. In some embodiments, attaching comprises ligating. In some embodiments, the first nucleic acid and the second nucleic acid are capped by a chemical modification to the 5′ and the 3′ ends. In some embodiments, the cap comprises a phosphorthioate. In some embodiments, the cap comprises a 2′ modified nucleoside, such as a 2′-O-modified ribose, a 2′-O-methyl nucleoside, or a 2′-O-methoxyethyl nucleoside. In some embodiments, the cap comprises an inverted dT modification. Additional methods of capping and protecting the ends of nucleic acids are provided elsewhere herein.

In some cases, the first nucleic acid capped with an adaptor having a size in a range from about 10 bp to about 1000 bp. In some cases, the second nucleic acid capped with an adaptor having a size in a range from about 10 bp to about 1000 bp. In some cases, the first nucleic acid capped with an adaptor having a size in a range from about 25 bp to about 1000 bp. In some cases, the second nucleic acid capped with an adaptor having a size in a range from about 25 bp to about 1000 bp. In some cases, the first nucleic acid capped with an adaptor having a size in a range from about 50 bp to about 1000 bp. In some cases, the second nucleic acid capped with an adaptor having a size in a range from about 50 bp to about 1000 bp. In some cases, the first nucleic acid capped with an adaptor having a size in a range from about 50 bp to about 200 bp. In some cases, the second nucleic acid capped with an adaptor having a size in a range from about 50 to about 200 bp. In some cases, the first nucleic acid capped with an adaptor having a size in a range from about 25 bp to about 200 bp. In some cases, the second nucleic acid capped with an adaptor having a size in a range from about 25 bp to about 200 bp. In some cases, the first nucleic acid capped with an adaptor having a size in a range from about 10 bp to about 200 bp. In some cases, the second nucleic acid capped with an adaptor having a size in a range from about 10 bp to about 200 bp. Smaller adapters are also consistent with the disclosure herein. Many adapters share a property that, when attached to a nucleic acid fragment, they convey exonuclease resistance to the nucleic acid. In some embodiments, the adapter is a modified nanopore adapter.

Provided herein are methods, compositions and kits related to the selective enrichment of nucleic acid of interest from a sample comprising a first nucleic acid and a second nucleic acid, wherein the second nucleic acid is the nucleic acid of interest. Also provided herein are methods for the selective exclusion from a sequencing reaction or from a sequence data set of the first nucleic acid. In some embodiments, the first nucleic acid comprises sequence encoding ribosomal RNA (rRNA), sequence encoding globin proteins, sequencing encoding a transposon, sequence encoding retroviral sequence, sequence comprising telomere sequence, sequence comprising sub-telomeric repeats, sequence comprising centromeric sequence, sequence comprising intron sequence, sequence comprising Alu repeats, SINE repeats, LINE repeats, dinucleic acid repeats, trinucleic acid repeats, tetranucleic acid repeats, poly-A repeats, poly-T repeats, poly-C repeats, poly-G repeats, AT-rich sequence, or GC-rich sequence.

In some cases, the first nucleic acid comprises sequence reverse-transcribed from RNA encoding ribosomal RNA, RNA encoding globins, RNA encoding overexpressed transcripts, or RNA that is otherwise disproportionately present or redundantly present in a sample.

In some embodiments a first nucleic acid is targeted, for example, using an endonuclease having a moiety that specifically binds to the first nucleic acid sequence. In some embodiments, a plurality of moieties includes members that bind to 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% of the first nucleic acid (e.g., host nucleic acid).

In some embodiments, a plurality of moieties includes members that bind to 1%-100%, 2%-100%, 3%-100%, 4%-100%, 5%-100%, 6%-100%, 7%-100%, 8%-100%, 9%-100%, 10%-100%, 11%-100%, 12%-100%, 13%-100%, 14%-100%, 15%-100%, 16%-100%, 17%-100%, 18%-100%, 19%-100%, 20%-100%, 21%-100%, 22%-100%, 23%-100%, 24%-100%, 25%-100%, 26%-100%, 27%-100%, 28%-100%, 29%-100%, 30%-100%, 31%-100%, 32%-100%, 33%-100%, 34%-100%, 35%-100%, 36%-100%, 37%-100%, 38%-100%, 39%-100%, 40%-100%, 41%-100%, 42%-100%, 43%-100%, 44%-100%, 45%-100%, 46%-100%, 47%-100%, 48%-100%, 49%-100%, 50%-100%, 51%-100%, 52%-100%, 53%-100%, 54%-100%, 55%-100%, 56%-100%, 57%-100%, 58%-100%, 59%-100%, 60%-100%, 61%-100%, 62%-100%, 63%-100%, 64%-100%, 65%-100%, 66%-100%, 67%-100%, 68%-100%, 69%-100%, 70%-100%, 71%-100%, 72%-100%, 73%-100%, 74%-100%, 75%-100%, 76%-100%, 77%-100%, 78%-100%, 79%-100%, 80%-100%, 81%-100%, 82%-100%, 83%-100%, 84%-100%, 85%-100%, 86%-100%, 87%-100%, 88%-100%, 89%-100%, 90%-100%, 91%-100%, 92%-100%, 93%-100%, 94%-100%, 95%-100%, 96%-100%, 97%-100%, 98%-100%, 99%-100% or 100% of the first nucleic acid (e.g., host nucleic acid).

In some embodiments, a plurality of moieties includes members that bind to 1%, 1%-2%, 1%-3%, 1%-4%, 1%-5%, 1%-6%, 1%-7%, 1%-8%, 1%-9%, 1%-10%, 1%-11%, 1%-12%, 1%-13%, 1%-14%, 1%-15%, 1%-16%, 1%-17%, 1%-18%, 1%-19%, 1%-20%, 1%-21%, 1%-22%, 1%-23%, 1%-24%, 1%-25%, 1%-26%, 1%-27%, 1%-28%, 1%-29%, 1%-30%, 1%-31%, 1%-32%, 1%-33%, 1%-34%, 1%-35%, 1%-36%, 1%-37%, 1%-38%, 1%-39%, 1%-40%, 1%-41%, 1%-42%, 1%-43%, 1%-44%, 1%-45%, 1%-46%, 1%-47%, 1%-48%, 1%-49%, 1%-50%, 1%-51%, 1%-52%, 1%-53%, 1%-54%, 1%-55%, 1%-56%, 1%-57%, 1%-58%, 1%-59%, 1%-60%, 1%-61%, 1%-62%, 1%-63%, 1%-64%, 1%-65%, 1%-66%, 1%-67%, 1%-68%, 1%-69%, 1%-70%, 1%-71%, 1%-72%, 1%-73%, 1%-74%, 1%-75%, 1%-76%, 1%-77%, 1%-78%, 1%-79%, 1%-80%, 1%-81%, 1%-82%, 1%-83%, 1%-84%, 1%-85%, 1%-86%, 1%-87%, 1%-88%, 1%-89%, 1%-90%, 1%-91%, 1%-92%, 1%-93%, 1%-94%, 1%-95%, 1%-96%, 1%-97%, 1%-98%, 1%-99% or 100% of the first nucleic acid (e.g., host nucleic acid).

In some embodiments the first nucleic acid comprises 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more than 99% of the total nucleic acids in the sample.

In some embodiments, the sample is a human genomic DNA sample. In some embodiments, the first nucleic acid comprises 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, or more than 70% of a sample. In some embodiments first nucleic acid comprises ⅔ or about ⅔ of a sample. In some embodiments the first nucleic acid comprises ⅔ of a sample.

In some embodiments a moiety that specifically binds to the first nucleic acid comprises a restriction endonuclease, such as a specific endonuclease that binds and cleaves at a recognition site that is specific to the first nucleic acid. In some embodiments a population of moieties that specifically bind to the first nucleic acid comprises at least one restriction endonuclease, two restriction endonucleases or more than two restriction endonucleases.

In some embodiments a moiety that specifically binds to the first nucleic acid comprises a guide RNA molecule. In some embodiments a population of moieties that specifically bind to first nucleic acid comprises a population of guide RNA molecules, such as a population of guide molecules that bind to the first nucleic acid.

### Endonuclease for Targeted Cleavage of Nucleic Acid

Methods disclosed herein comprise targeting cleavage of the first nucleic acid using a site-specific, targetable, and/or engineered nuclease or nuclease system. Such nucleases may create double-stranded break (DSBs) at desired locations in a genomic, cDNA or other nucleic acid molecule. In other examples, a nuclease may create a single strand break. In some cases, two nucleases are used, each of which generates a single strand break. Many cleavage enzymes consistent with the disclosure herein share a trait that they yield molecules having an end accessible for single stranded or double stranded exonuclease activity.

The endonuclease used herein can be a restriction enzyme specific to at least one site on the first nucleic acid and that does not cleave a second nucleic acid. The endonuclease described herein can be specific to a repetitive nucleic sequence in a host genome, such as a transposon or other repeat, a centromeric region, or other repeat sequence. For example, some restriction endonucleases consistent with the disclosure herein are Alu specific restriction enzymes. A restriction is Alu specific or, for that matter, other target ‘specific’ if it cuts a target and does not cut other substrates, or cuts other targets infrequently so as to differentially deplete its ‘specific’ target. The presence of a non-Alu or other non-target cleavage, such as due to the rare occurrence of the cleavage site elsewhere in a host genome or transcriptome, or in a pathogen or other rare nucleic acid present in a sample, does not render an endonuclease ‘nonspecific’ so long as differential depletion of undesired nucleic acid is effected.

The first nucleic acid can include a restriction enzyme Alu recognition site. The second nucleic acid does not include the Alu recognition site. In some embodiments, the first nucleic acid comprises at least one sequence that maps to at least one nucleic acid recognition site selected from the group consisting of recognition sites of AluI, AsuHPI, Bpu10I, BssECI, BstDEI, BstMAI, HinfI, and BstTUI. In some embodiments, the second nucleic acid does not include at least one of the recognition sites selected from recognition sites of AluI, AsuHPI, Bpu10I, BssECI, BstDEI, BstMAI, HinfI, and BstTUI.

Endonucleases consistent with the disclosure herein variously include at least one selected from Clustered Regulatory Interspaced Short Palindromic Repeat (CRISPR)/Cas system protein-gRNA complexes, Zinc Finger Nucleases (ZFN), and Transcription activator like effector nucleases. In some embodiments, the gRNAs are complementary to at least one site on the first nucleic acid to generate cleaved first nucleic acids capped only on one end. Other programmable, nucleic acid sequence specific endonucleases are also consistent with the disclosure herein.

Engineered nucleases such as zinc finger nucleases (ZFNs), Transcription Activator-Like Effector Nucleases (TALENs), engineered homing endonucleases, and RNA or DNA guided endonucleases, such as CRISPR/Cas such as Cas9 or CPF1, and/or Argonaute systems, are particularly appropriate to carry out some of the methods of the present disclosure. Additionally or alternatively, RNA targeting systems may be used, such as CRISPR/Cas systems including c2c2 nucleases.

Methods disclosed herein may comprise cleaving a target nucleic acid using CRISPR systems, such as a Type I, Type II, Type III, Type IV, Type V, or Type VI CRISPR system. CRISPR/Cas systems may be multi-protein systems or single effector protein systems. Multi-protein, or Class 1, CRISPR systems include Type I, Type III, and Type IV systems. Alternatively, Class 2 systems include a single effector molecule and include Type II, Type V, and Type VI.

CRISPR systems used in some methods disclosed herein may comprise a single or multiple effector proteins. An effector protein may comprise one or multiple nuclease domains. An effector protein may target DNA or RNA, and the DNA or RNA may be single stranded or double stranded. Effector proteins may generate double strand or single strand breaks. Effector proteins may comprise mutations in a nuclease domain thereby generating a nickase protein. Effector proteins may comprise mutations in one or more nuclease domains, thereby generating a catalytically dead nuclease that is able to bind but not cleave a target sequence. CRISPR systems may comprise a single or multiple guiding RNAs. The gRNA may comprise a crRNA. The gRNA may comprise a chimeric RNA with crRNA and tracrRNA sequences. The gRNA may comprise a separate crRNA and tracrRNA. Target nucleic acid sequences may comprise a protospacer adjacent motif (PAM) or a protospacer flanking site (PFS). The PAM or PFS may be 3′ or 5′ of the target or protospacer site. Cleavage of a target sequence may generate blunt ends, 3′ overhangs, or 5′ overhangs. In some cases, target nucleic acids do not comprise a PAM or PFS.

A gRNA may comprise a spacer sequence. Spacer sequences may be complementary to target sequences or protospacer sequences. Spacer sequences may be 10, 11, 12, 13, 14, 15, 16, 17, 18, 19, 20, 21, 22, 23, 24, 25, 26, 27, 28, 29, 30, 31, 32, 33, 34, 35, or 36 nucleotides in length. In some examples, the spacer sequence may be less than 10 or more than 36 nucleotides in length.

A gRNA may comprise a repeat sequence. In some cases, the repeat sequence is part of a double stranded portion of the gRNA. A repeat sequence may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some examples, the spacer sequence may be less than 10 or more than 50 nucleotides in length.

A gRNA may comprise one or more synthetic nucleotides, non-naturally occurring nucleotides, nucleotides with a modification, deoxyribonucleotide, or any combination thereof. Additionally or alternatively, a gRNA may comprise a hairpin, linker region, single stranded region, double stranded region, or any combination thereof. Additionally or alternatively, a gRNA may comprise a signaling or reporter molecule.

A CRISPR nuclease may be endogenously or recombinantly expressed. A CRISPR nuclease may be encoded on a chromosome, extrachromosomally, or on a plasmid, synthetic chromosome, or artificial chromosome. A CRISPR nuclease may be provided as a polypeptide or mRNA encoding the polypeptide. In such examples, polypeptide or mRNA may be delivered through standard mechanisms known in the art, such as through the use of cell permeable peptides, nanoparticles, or viral particles.

gRNAs may be encoded by genetic or episomal DNA. gRNAs may be provided or delivered concomitantly with a CRISPR nuclease or sequentially. Guide RNAs may be chemically synthesized, in vitro transcribed or otherwise generated using standard RNA generation techniques known in the art.

A CRISPR system may be a Type II CRISPR system, for example a Cas9 system. The Type II nuclease may comprise a single effector protein, which, in some cases, comprises a RuvC and HNH nuclease domains. In some cases a functional Type II nuclease may comprise two or more polypeptides, each of which comprises a nuclease domain or fragment thereof. The target nucleic acid sequences may comprise a 3′ protospacer adjacent motif (PAM). In some examples, the PAM may be 5′ of the target nucleic acid. Guide RNAs (gRNA) may comprise a single chimeric gRNA, which contains both crRNA and tracrRNA sequences. Alternatively, the gRNA may comprise a set of two RNAs, for example a crRNA and a tracrRNA. The Type II nuclease may generate a double strand break, which is some cases creates two blunt ends. In some cases, the Type II CRISPR nuclease is engineered to be a nickase such that the nuclease only generates a single strand break. In such cases, two distinct nucleic acid sequences may be targeted by gRNAs such that two single strand breaks are generated by the nickase. In some examples, the two single strand breaks effectively create a double strand break. In some cases where a Type II nickase is used to generate two single strand breaks, the resulting nucleic acid free ends may either be blunt, have a 3′ overhang, or a 5′ overhang. In some examples, a Type II nuclease may be catalytically dead such that it binds to a target sequence, but does not cleave. For example, a Type II nuclease may have mutations in both the RuvC and HNH domains, thereby rendering the both nuclease domains non-functional. A Type II CRISPR system may be one of three sub-types, namely Type II-A, Type II-B, or Type II-C.

A CRISPR system may be a Type V CRISPR system, for example a Cpf1, C2c1, or C2c3 system. The Type V nuclease may comprise a single effector protein, which in some cases comprises a single RuvC nuclease domain. In other cases, a function Type V nuclease comprises a RuvC domain split between two or more polypeptides. In such cases, the target nucleic acid sequences may comprise a 5′ PAM or 3′ PAM. Guide RNAs (gRNA) may comprise a single gRNA or single crRNA, such as may be the case with Cpf1. In some cases, a tracrRNA is not needed. In other examples, such as when C2c1 is used, a gRNA may comprise a single chimeric gRNA, which contains both crRNA and tracrRNA sequences or the gRNA may comprise a set of two RNAs, for example a crRNA and a tracrRNA. The Type V CRISPR nuclease may generate a double strand break, which in some cases generates a 5′ overhang. In some cases, the Type V CRISPR nuclease is engineered to be a nickase such that the nuclease only generates a single strand break. In such cases, two distinct nucleic acid sequences may be targeted by gRNAs such that two single strand breaks are generated by the nickase. In some examples, the two single strand breaks effectively create a double strand break. In some cases where a Type V nickase is used to generate two single strand breaks, the resulting nucleic acid free ends may either be blunt, have a 3′ overhang, or a 5′ overhang. In some examples, a Type V nuclease may be catalytically dead such that it binds to a target sequence, but does not cleave. For example, a Type V nuclease could have mutations a RuvC domain, thereby rendering the nuclease domain non-functional.

A CRISPR system may be a Type VI CRISPR system, for example a C2c2 system. A Type VI nuclease may comprise a HEPN domain. In some examples, the Type VI nuclease comprises two or more polypeptides, each of which comprises a HEPN nuclease domain or fragment thereof. In such cases, the target nucleic acid sequences may by RNA, such as single stranded RNA. When using Type VI CRISPR system, a target nucleic acid may comprise a protospacer flanking site (PFS). The PFS may be 3′ or 5′ or the target or protospacer sequence. Guide RNAs (gRNA) may comprise a single gRNA or single crRNA. In some cases, a tracrRNA is not needed. In other examples, a gRNA may comprise a single chimeric gRNA, which contains both crRNA and tracrRNA sequences or the gRNA may comprise a set of two RNAs, for example a crRNA and a tracrRNA. In some examples, a Type VI nuclease may be catalytically dead such that it binds to a target sequence, but does not cleave. For example, a Type VI nuclease may have mutations in a HEPN domain, thereby rendering the nuclease domains non-functional.

Non-limiting examples of suitable nucleases, including nucleic acid-guided nucleases, for use in the present disclosure include C2c1, C2c2, C2c3, Cas1, Cas1B, Cas2, Cas3, Cas4, Cas5, Cas6, Cas7, Cas8, Cas9 (also known as Csn1 and Csx12), Cas10, Cpf1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx100, Csx16, CsaX, Csx3, Csx1, Csx15, Csf1, Csf2, Csf3, Csf4, homologues thereof, orthologues thereof, or modified versions thereof.

In some methods disclosed herein, Argonaute (Ago) systems may be used to cleave target nucleic acid sequences. Ago protein may be derived from a prokaryote, eukaryote, or archaea. The target nucleic acid may be RNA or DNA. A DNA target may be single stranded or double stranded. In some examples, the target nucleic acid does not require a specific target flanking sequence, such as a sequence equivalent to a protospacer adjacent motif or protospacer flanking sequence. The Ago protein may create a double strand break or single strand break. In some examples, when a Ago protein forms a single strand break, two Ago proteins may be used in combination to generate a double strand break. In some examples, an Ago protein comprises one, two, or more nuclease domains. In some examples, an Ago protein comprises one, two, or more catalytic domains. One or more nuclease or catalytic domains may be mutated in the Ago protein, thereby generating a nickase protein capable of generating single strand breaks. In other examples, mutations in one or more nuclease or catalytic domains of an Ago protein generates a catalytically dead Ago protein that may bind but not cleave a target nucleic acid.

Ago proteins may be targeted to target nucleic acid sequences by a guiding nucleic acid. In many examples, the guiding nucleic acid is a guide DNA (gDNA). The gDNA may have a 5′ phosphorylated end. The gDNA may be single stranded or double stranded. Single stranded gDNA may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some examples, the gDNA may be less than 10 nucleotides in length. In some examples, the gDNA may be more than 50 nucleotides in length.

Argonaute-mediated cleavage may generate blunt end, 5′ overhangs, or 3′ overhangs. In some examples, one or more nucleotides are removed from the target site during or following cleavage.

Argonaute protein may be endogenously or recombinantly expressed. Argonaute may be encoded on a chromosome, extrachromosomally, or on a plasmid, synthetic chromosome, or artificial chromosome. Additionally or alternatively, an Argonaute protein may be provided as a polypeptide or mRNA encoding the polypeptide. In such examples, polypeptide or mRNA may be delivered through standard mechanisms known in the art, such as through the use of peptides, nanoparticles, or viral particles.

Guide DNAs may be provided by genetic or episomal DNA. In some examples, gDNA are reverse transcribed from RNA or mRNA. In some examples, guide DNAs may be provided or delivered concomitantly with an Ago protein or sequentially. Guide DNAs may be chemically synthesized, assembled, or otherwise generated using standard DNA generation techniques known in the art. Guide DNAs may be cleaved, released, or otherwise derived from genomic DNA, episomal DNA molecules, isolated nucleic acid molecules, or any other source of nucleic acid molecules.

Nuclease fusion proteins may be recombinantly expressed. A nuclease fusion protein may be encoded on a chromosome, extrachromosomally, or on a plasmid, synthetic chromosome, or artificial chromosome. A nuclease and a chromatin-remodeling enzyme may be engineered separately, and then covalently linked. A nuclease fusion protein may be provided as a polypeptide or mRNA encoding the polypeptide. In such examples, polypeptide or mRNA may be delivered through standard mechanisms known in the art, such as through the use of peptides, nanoparticles, or viral particles.

A guide nucleic acid may complex with a compatible nucleic acid-guided nuclease and may hybridize with a target sequence, thereby directing the nuclease to the target sequence. A subject nucleic acid-guided nuclease capable of complexing with a guide nucleic acid may be referred to as a nucleic acid-guided nuclease that is compatible with the guide nucleic acid. Likewise, a guide nucleic acid capable of complexing with a nucleic acid-guided nuclease may be referred to as a guide nucleic acid that is compatible with the nucleic acid-guided nucleases.

A guide nucleic acid may be DNA. A guide nucleic acid may be RNA. A guide nucleic acid may comprise both DNA and RNA. A guide nucleic acid may comprise modified of non-naturally occurring nucleotides. In cases where the guide nucleic acid comprises RNA, the RNA guide nucleic acid may be encoded by a DNA sequence on a polynucleotide molecule such as a plasmid, linear construct, or editing cassette as disclosed herein.

A guide nucleic acid may comprise a guide sequence. A guide sequence is a polynucleotide sequence having sufficient complementarity with a target polynucleotide sequence to hybridize with the target sequence and direct sequence-specific binding of a complexed nucleic acid-guided nuclease to the target sequence. The degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences. In some aspects, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some aspects, a guide sequence is less than about 75, 50, 45, 40, 35, 30, 25, 20 nucleotides in length. Preferably the guide sequence is 10-30 nucleotides long. The guide sequence may be 10-25 nucleotides in length. The guide sequence may be 10-20 nucleotides in length. The guide sequence may be 15-30 nucleotides in length. The guide sequence may be 20-30 nucleotides in length. The guide sequence may be 15-25 nucleotides in length. The guide sequence may be 15-20 nucleotides in length. The guide sequence may be 20-25 nucleotides in length. The guide sequence may be 22-25 nucleotides in length. The guide sequence may be 15 nucleotides in length. The guide sequence may be 16 nucleotides in length. The guide sequence may be 17 nucleotides in length. The guide sequence may be 18 nucleotides in length. The guide sequence may be 19 nucleotides in length. The guide sequence may be 20 nucleotides in length. The guide sequence may be 21 nucleotides in length. The guide sequence may be 22 nucleotides in length. The guide sequence may be 23 nucleotides in length. The guide sequence may be 24 nucleotides in length. The guide sequence may be 25 nucleotides in length.

A guide nucleic acid may comprise a scaffold sequence. In general, a “scaffold sequence” includes any sequence that has sufficient sequence to promote formation of a targetable nuclease complex, wherein the targetable nuclease complex comprises a nucleic acid-guided nuclease and a guide nucleic acid comprising a scaffold sequence and a guide sequence. Sufficient sequence within the scaffold sequence to promote formation of a targetable nuclease complex may include a degree of complementarity along the length of two sequence regions within the scaffold sequence, such as one or two sequence regions involved in forming a secondary structure. In some cases, the one or two sequence regions are comprised or encoded on the same polynucleotide. In some cases, the one or two sequence regions are comprised or encoded on separate polynucleotides. Optimal alignment may be determined by any suitable alignment algorithm, and may further account for secondary structures, such as self-complementarity within either the one or two sequence regions. In some aspects, the degree of complementarity between the one or two sequence regions along the length of the shorter of the two when optimally aligned is about or more than about 25%, 30%, 40%, 50%, 60%, 70%, 80%, 90%, 95%, 97.5%, 99%, or higher. In some aspects, at least one of the two sequence regions is about or more than about 5, 6, 7, 8, 9, 10, 11, 12, 13, 14, 15, 16, 17, 18, 19, 20, 21, 22, 23, 24, 25, 30, 40, 50, or more nucleotides in length. In some aspects, at least one of the two sequence regions is about 10-30 nucleotides in length. At least one of the two sequence regions may be 10-25 nucleotides in length. At least one of the two sequence regions may be 10-20 nucleotides in length. At least one of the two sequence regions may be 15-30 nucleotides in length. At least one of the two sequence regions may be 20-30 nucleotides in length. At least one of the two sequence regions may be 15-25 nucleotides in length. At least one of the two sequence regions may be 15-20 nucleotides in length. At least one of the two sequence regions may be 20-25 nucleotides in length. At least one of the two sequence regions may be 22-25 nucleotides in length. At least one of the two sequence regions may be 15 nucleotides in length. At least one of the two sequence regions may be 16 nucleotides in length. At least one of the two sequence regions may be 17 nucleotides in length. At least one of the two sequence regions may be 18 nucleotides in length. At least one of the two sequence regions may be 19 nucleotides in length. At least one of the two sequence regions may be 20 nucleotides in length. At least one of the two sequence regions may be 21 nucleotides in length. At least one of the two sequence regions may be 22 nucleotides in length. At least one of the two sequence regions may be 23 nucleotides in length. At least one of the two sequence regions may be 24 nucleotides in length. At least one of the two sequence regions may be 25 nucleotides in length.

A scaffold sequence of a subject guide nucleic acid may comprise a secondary structure. A secondary structure may comprise a pseudoknot region. In some example, the compatibility of a guide nucleic acid and nucleic acid-guided nuclease is at least partially determined by sequence within or adjacent to a pseudoknot region of the guide RNA. In some cases, binding kinetics of a guide nucleic acid to a nucleic acid-guided nuclease is determined in part by secondary structures within the scaffold sequence. In some cases, binding kinetics of a guide nucleic acid to a nucleic acid-guided nuclease is determined in part by nucleic acid sequence with the scaffold sequence.

In aspects of the disclosure the terms “guide nucleic acid” refers to a polynucleotide comprising 1) a guide sequence capable of hybridizing to a target sequence and 2) a scaffold sequence capable of interacting with or complexing with a nucleic acid-guided nuclease as described herein.

A guide nucleic acid may be compatible with a nucleic acid-guided nuclease when the two elements may form a functional targetable nuclease complex capable of cleaving a target sequence. Often, a compatible scaffold sequence for a compatible guide nucleic acid may be found by scanning sequences adjacent to native nucleic acid-guided nuclease loci. In other words, native nucleic acid-guided nucleases may be encoded on a genome within proximity to a corresponding compatible guide nucleic acid or scaffold sequence.

Nucleic acid-guided nucleases may be compatible with guide nucleic acids that are not found within the nucleases endogenous host. Such orthogonal guide nucleic acids may be determined by empirical testing. Orthogonal guide nucleic acids may come from different bacterial species or be synthetic or otherwise engineered to be non-naturally occurring.

Orthogonal guide nucleic acids that are compatible with a common nucleic acid-guided nuclease may comprise one or more common features. Common features may include sequence outside a pseudoknot region. Common features may include a pseudoknot region. Common features may include a primary sequence or secondary structure.

A guide nucleic acid may be engineered to target a desired target sequence by altering the guide sequence such that the guide sequence is complementary to the target sequence, thereby allowing hybridization between the guide sequence and the target sequence. A guide nucleic acid with an engineered guide sequence may be referred to as an engineered guide nucleic acid. Engineered guide nucleic acids are often non-naturally occurring and are not found in nature.

In some embodiments the guide RNA molecule interferes with sequencing directly, for example by binding the target sequence to prevent nucleic acid polymerization to occur across the bound sequence. In some embodiments the guide RNA molecule works in tandem with a RNA-DNA hybrid binding moiety such as a protein. In some embodiments the guide RNA molecule directs modification of member of the sequencing library to which it may bind, such as methylation, base excision, or cleavage, such that in some embodiments the member of the sequencing library to which it is bound becomes unsuitable for further sequencing reactions. In some embodiments, the guide RNA molecule directs endonucleolytic cleavage of the DNA molecule to which it is bound, for example by a protein having endonuclease activity such as Cas9 protein. Zinc Finger Nucleases (ZFN), Transcription activator like effector nucleases and Clustered Regulatory Interspaced Short Palindromic Repeat/Cas based RNA guided DNA nuclease (CRISPR/Cas9), among others, are compatible with some embodiments of the disclosure herein.

A guide RNA molecule comprises sequence that base-pairs with target sequence that is to be removed from sequencing (the first nucleic acid). In some embodiments the base-pairing is complete, while in some embodiments the base pairing is partial or comprises bases that are unpaired along with bases that are paired to non-target sequence.

A guide RNA may comprise a region or regions that form an RNA ‘hairpin’ structure. Such region or regions comprise partially or completely palindromic sequence, such that 5′ and 3′ ends of the region may hybridize to one another to form a double-strand ‘stem’ structure, which in some embodiments is capped by a non-palindromic loop tethering each of the single strands in the double strand loop to one another.

In some embodiments the Guide RNA comprises a stem loop such as a tracrRNA stem loop. A stem loop such as a tracrRNA stem loop may complex with or bind to a nucleic acid endonuclease such as Cas9 DNA endonuclease. Alternately, a stem loop may complex with an endonuclease other than Cas9 or with a nucleic acid modifying enzyme other than an endonuclease, such as a base excision enzyme, a methyltransferase, or an enzyme having other nucleic acid modifying activity that interferes with one or more DNA polymerase enzymes.

The tracrRNA/CRISPR/Endonuclease system was identified as an adaptive immune system in eubacterial and archaeal prokaryotes whereby cells gain resistance to repeated infection by a virus of a known sequence. See, for example, Deltcheva E, Chylinski K, Sharma C M, Gonzales K, Chao Y, Pirzada Z A et al. (2011) “CRISPR RNA maturation by trans-encoded small RNA and host factor RNase III” Nature 471 (7340): 602-7. doi:10.1038/nature09886. PMC 3070239. PMID 21455174; Terns M P, Terns R M (2011) “CRISPR-based adaptive immune systems” Curr Opin Microbiol 14 (3): 321-7. doi:10.1016/j.mib.2011.03.005. PMC 3119747. PMID 21531607; Jinek M, Chylinski K, Fonfara I, Hauer M, Doudna J A, Charpentier E (2012) “A Programmable Dual-RNA-Guided DNA Endonuclease in Adaptive Bacterial Immunity” Science 337 (6096): 816-21. doi:10.1126/science.1225829. PMID 22745249; and Brouns S J (2012) “A swiss army knife of immunity” Science 337 (6096): 808-9. doi:10.1126/science.1227253. PMID 22904002. The system has been adapted to direct targeted mutagenesis in eukaryotic cells. See, e.g., Wenzhi Jiang, Huanbin Zhou, Honghao Bi, Michael Fromm, Bing Yang, and Donald P. Weeks (2013) “Demonstration of CRISPR/Cas9/sgRNA-mediated targeted gene modification in Arabidopsis, tobacco, sorghum and rice” Nucleic Acids Res. November 2013; 41(20): e188, Published online Aug. 31, 2013. doi: 10.1093/nar/gkt780, and references therein.

As contemplated herein, guide RNA are used in some embodiments to provide sequence specificity to a DNA endonuclease such as a Cas9 endonuclease. In these embodiments a guide RNA comprises a hairpin structure that binds to or is bound by an endonuclease such as Cas9 (other endonucleases are contemplated as alternatives or additions in some embodiments), and a guide RNA further comprises a recognition sequence that binds to or specifically binds to or exclusively binds to a sequence that is to be removed from a sequencing library or a sequencing reaction. The length of the recognition sequence in a guide RNA may vary according to the degree of specificity desired in the sequence elimination process. Short recognition sequences, comprising frequently occurring sequence in the sample or comprising differentially abundant sequence (abundance of AT in an AT-rich genome sample or abundance of GC in a GC-rich genome sample) are likely to identify a relatively large number of sites and therefore to direct frequent nucleic acid modification such as endonuclease activity, base excision, methylation or other activity that interferes with at least one DNA polymerase activity. Long recognition sequences, comprising infrequently occurring sequence in the sample or comprising underrepresented base combinations (abundance of GC in an AT-rich genome sample or abundance of AT in a GC-rich genome sample) are likely to identify a relatively small number of sites and therefore to direct infrequent nucleic acid modification such as endonuclease activity, base excision, methylation or other activity that interferes with at least one DNA polymerase activity. Accordingly, as disclosed herein, in some embodiments one may regulate the frequency of sequence removal from a sequence reaction through modifications to the length or content of the recognition sequence.

Guide RNA may be synthesized through a number of methods consistent with the disclosure herein. Standard synthesis techniques may be used to produce massive quantities of guide RNAs, and/or for highly-repetitive targeted regions, which may require only a few guide RNA molecules to target a multitude of unwanted loci. The double stranded DNA molecules can comprise an RNA site specific binding sequence, a guide RNA sequence for Cas9 protein and a T7 promoter site. In some cases, the double stranded DNA molecules can be less than about 100 bp length. T7 polymerase can be used to create the single stranded RNA molecules, which may include the target RNA sequence and the guide RNA sequence for the Cas9 protein.

Guide RNA sequences may be designed through a number of methods. For example, in some embodiments, non-genic repeat sequences of the human genome are broken up into, for example, 100 bp sliding windows. Double stranded DNA molecules can be synthesized in parallel on a microarray using photolithography.

The windows may vary in size. 30-mer target sequences can be designed with a short trinucleotide protospacer adjacent motif (PAM) sequence of N-G-G flanking the 5′ end of the target design sequence, which in some cases facilitates cleavage. See, among others, Giedrius Gasiunas et al., (2012) “Cas9-crRNA ribonucleoprotein complex mediates specific DNA cleavage for adaptive immunity in bacteria” Proc. Natl. Acad. Sci. USA. September 25, 109(39): E2579-E2586, which is hereby incorporated by reference in its entirety. Redundant sequences can be eliminated and the remaining sequences can be analyzed using a search engine (e.g. BLAST) against the human genome to avoid hybridization against REFSEQ, ENSEMBL and other gene databases to avoid nuclease activity at these sites. The universal Cas9 tracer RNA sequence can be added to the guide RNA target sequence and then flanked by the T7 promoter. The sequences upstream of the T7 promoter site can be synthesized. Due to the highly repetitive nature of the target regions in the human genome, in many embodiments, a relatively small number of guide RNA molecules will digest a larger percentage of NGS library molecules.

Although only about 50% of protein coding genes are estimated to have exons comprising the NGG PAM (photospacer adjacent motif) sequence, multiple strategies are provided herein to increase the percentage of the genome that can be targeted with the Cas9 cutting system. For example, if a PAM sequence is not available in a DNA region, a PAM sequence may be introduced via a combination strategy using a guide RNA coupled with a helper DNA comprising the PAM sequence. The helper DNA can be synthetic and/or single stranded. The PAM sequence in the helper DNA will not be complimentary to the gDNA knockout target in the NGS library, and may therefore be unbound to the target NGS library template, but it can be bound to the guide RNA. The guide RNA can be designed to hybridize to both the target sequence and the helper DNA comprising the PAM sequence to form a hybrid DNA:RNA:DNA complex that can be recognized by the Cas9 system.

The PAM sequence may be represented as a single stranded overhang or a hairpin. The hairpin can, in some cases, comprise modified nucleotides that may optionally be degraded. For example, the hairpin can comprise Uracil, which can be degraded by Uracil DNA Glycosylase.

As an alternative to using a DNA comprising a PAM sequence, modified Cas9 proteins without the need of a PAM sequence or modified Cas9 with lower sensitivity to PAM sequences may be used without the need for a helper DNA sequence.

In further cases, the guide RNA sequence used for Cas9 recognition may be lengthened and inverted at one end to act as a dual cutting system for close cutting at multiple sites. The guide RNA sequence can produce two cuts on a NGS DNA library target. This can be achieved by designing a single guide RNA to alternate strands within a restricted distance. One end of the guide RNA may bind to the forward strand of a double stranded DNA library and the other may bind to the reverse strand. Each end of the guide RNA can comprise the PAM sequence and a Cas9 binding domain. This may result in a dual double stranded cut of the NGS library molecules from the same DNA sequence at a defined distance apart.

Alternative versions of the assay comprise at least one sequence-specific nuclease, and in some cases a combination of sequence-specific nucleases, such as at least one restriction endonuclease having a recognition site that is abundant in the first nucleic acid. In some cases an enzyme comprises an activity that yields double-stranded breaks in response to a specific sequence. In some cases an enzyme comprises any nuclease or other enzyme that digests double-stranded nucleic acid material in RNA/DNA hybrids.

Nucleic acid probes (e.g. biotinylated probes) complementary to the second nucleic acids can be hybridized to the second nucleic acids in solution and pulled down with, e.g., magnetic streptavidin-coated beads. Non bound nucleic acids can be washed away and the captured nucleic acids may then be eluted and amplified for sequencing or genotyping.

In some embodiments, practice of the methods herein reduces the sequencing time duration of a sequencing reaction, such that a nucleic acid library is sequenced in a shorter time, or using fewer reagents, or using less computing power. In some embodiments, practice of the methods herein reduces the sequencing time duration of a sequencing reaction for a given nucleic acid library to about 90%, 80%, 70%, 60%, 50%, 40%, 33%, 30% or less than 30% of the time required to sequence the library in the absence of the practice of the methods herein.

In some embodiments, a specific read sequence from a specific region is of particular interest in a given sequencing reaction. Measures to allow the rapid identification of such a specific region are beneficial as they may decrease computation time or reagent requirements or both computation time and reagent requirements.

Some embodiments relate to the generation of guide RNA molecules. Guide RNA molecules are in some cases transcribed from DNA templates. A number of RNA polymerases may be used, such as T7 polymerase, RNA PolI, RNA PolII, RNA PolIII, an organellar RNA polymerase, a viral RNA polymerase, or a eubacterial or archaeal polymerase. In some cases the polymerase is T7.

Guide RNA generating templates comprise a promoter, such as a promoter compatible with transcription directed by T7 polymerase, RNA PolI, RNA PolII, RNA PolIII, an organellar RNA polymerase, a viral RNA polymerase, or a eubacterial or archaeal polymerase. In some cases the promoter is a T7 promoter.

Guide RNA templates encode a tag sequence in some cases. A tag sequence binds to a nucleic acid modifying enzyme such as a methylase, base excision enzyme or an endonuclease. In the context of a larger Guide RNA molecule bound to a nontarget site, a tag sequence tethers an enzyme to a nucleic acid nontarget region, directing activity to the nontarget site. An exemplary tethered enzyme is an endonuclease such as Cas9.

Guide RNA templates are complementary to the first nucleic acid corresponding to ribosomal RNA sequences, sequences encoding globin proteins, sequences encoding a transposon, sequences encoding retroviral sequences, sequences comprising telomere sequences, sequences comprising sub-telomeric repeats, sequences comprising centromeric sequences, sequences comprising intron sequences, sequences comprising Alu repeats, sequences comprising SINE repeats, sequences comprising LINE repeats, sequences comprising dinucleic acid repeats, sequences comprising trinucleic acid repeats, sequences comprising tetranucleic acid repeats, sequences comprising poly-A repeats, sequences comprising poly-T repeats, sequences comprising poly-C repeats, sequences comprising poly-G repeats, sequences comprising AT-rich sequences, or sequences comprising GC-rich sequences.

In many cases, the tag sequence comprises a stem-loop, such as a partial or total stem-loop structure. The ‘stem’ of the stem loop structure is encoded by a palindromic sequence in some cases, either complete or interrupted to introduce at least one ‘kink’ or turn in the stem. The ‘loop’ of the stem loop structure is not involved in stem base pairing in most cases. In some cases, the stem loop is encoded by a tracr sequence, such as a tracr sequence disclosed in references incorporated herein. Some stem loops bind, for example, Cas9 or other endonuclease.

Guide RNA molecules additionally comprise a recognition sequence. The recognition sequence is completely or incompletely reverse-complementary to a nontarget sequence to be eliminated from a nucleic acid library sequence set. As RNA is able to hybridize using base pair combinations (G:U base pairing, for example) that do not occur in DNA-DNA hybrids, the recognition sequence does not need to be an exact reverse complement of the nontarget sequence to bind. In addition, small perturbations from complete base pairing are tolerated in some cases.

### End Protection

Protecting the ends of DNA molecules from degradation can be effected through a number of approaches, provided that an end result is prevention of adapter-added fragments from exonuclease degradation at the site of adapter attachment. Adapters are added through ligation, polymerase mediated amplification, tagmentation via transposase delivery, end modification or other approaches. Representative adapters include hairpin adapters that effectively link the two strands of a double-stranded nucleic acid to form a single-stranded circular molecule if added at both ends. Such a molecule lacks an exposed end for single stranded or double stranded exonuclease degradation unless it is further cleaved by an endonuclease. Protection is also effected by attachment of an oligonucleotide or other molecule that is resistant to exonuclease activity. Examples of exonuclease-resistant adapters include phosphorthioate oligos, 2-O methyl modified nucleotide sugars, inverted dT or ddT, phosphorylation, C3 spacers or other modifications that inhibit an exonuclease from traversing the modification so as do degrade adjacent nucleic acids. Alternately or in combination, in some cases an ‘adapter’ constitutes modification to the ends of sample nucleic acids without ligation of additional molecules, such that the modification renders the nucleic acids resistant to exonuclease degradation.

A particular feature of the adapters herein is that, although they operate locally independent of one another, a nucleic acid is not protected from degradation unless both ends are subjected to adapter addition or modification. Otherwise, although and adapter-added end is protected from exonuclease activity, the opposite end of the nucleic acid is vulnerable to degradation such that the molecule as a whole is degraded. This is the fate of nucleic acids that are adapter modified but then cleaved by a sequence-specific nucleic acid endonuclease as contemplated herein, so as to yield at least two exposed, unprotected nucleic acid ends.

### Non-Host Nucleic Acids

Targeted depletion methods herein result in removal of a first nucleic acid and enrichment of a second nucleic acid from the sample. Said sample can be used to make a library for sequencing and said sequencing delivers sequence data that can be mostly derived from the second nucleic acid. For example, the second nucleic acid can be a non-host nucleic acid.

In certain aspects, provided herein are methods that result in enrichment of a microbial pathogen. In some cases, methods herein enable identification of said microbial pathogen. In some embodiments the microbial pathogen comprises a bacterial pathogen. In some embodiments, the bacterial pathogen is a Bacillus such as a Bacillus anthracis or a Bacillus cereus; a Bartonella such as a Bartonella henselae or a Bartonella quintana; a Bordetella such as a Bordetella pertussis; a Borrelia such as a Borrelia burgdorferi, a Borrelia garinii, a Borrelia afzelii, a Borrelia recurrentis; a Brucella such as a Brucella abortus, a Brucella canis, a Brucella melitensis or a Brucella suis; a Campylobacter such as a Campylobacter jejuni; a Chlamydia or Chlamydophila such as Chlamydia pneumoniae, Chlamydia trachomatis, Chlamydophila psittaci; a Clostridium such as a Clostridium botulinum, a Clostridium difficile, a Clostridium perfringens, a Clostridium tetani; a Corynebacterium such as a Corynebacterium diphtheriae; an Enterococcus such as a Enterococcus faecalis or a Enterococcus faecium; a Escherichia such as a Escherichia coli; a Francisella such as a Francisella tularensis; a Haemophilus such as a Haemophilus influenzae; a Helicobacter such as a Helicobacter pylori; a Legionella such as a Legionella pneumophila; a Leptospira such as a Leptospira interrogans, a Leptospira santarosai, a Leptospira weilii or a Leptospira noguchii; a Listeria such as a Listeria monocytogenes; a Mycobacterium such as a Mycobacterium leprae, a Mycobacterium tuberculosis or a Mycobacterium ulcerans; a Mycoplasma such as a Mycoplasma pneumoniae; a Neisseria such as a Neisseria gonorrhoeae or a Neisseria meningitidis; a Pseudomonas such as a Pseudomonas aeruginosa; a Rickettsia such as a Rickettsia rickettsii; a Salmonella such as a Salmonella typhi or a Salmonella typhimurium; a Shigella such as a Shigella sonnei; a Staphylococcus such as a Staphylococcus aureus, a Staphylococcus epidermidis, a Staphylococcus saprophyticus; a Streptococcus such as a Streptococcus agalactiae, a Streptococcus pneumoniae, a Streptococcus pyogenes; a Treponema such as a Treponema pallidum; a Vibrio such as a Vibrio cholerae; a Yersinia such as a Yersinia pestis, a Yersinia enterocolitica or a Yersinia pseudotuberculosis. In some embodiments, the microbial pathogen comprises a viral pathogen. In some embodiments, the viral pathogen comprises a Adenoviridae such as, an Adenovirus; a Herpesviridae such as a Herpes simplex, type 1, a Herpes simplex, type 2, a Varicella-zoster virus, an Epstein-barr virus, a Human cytomegalovirus, a Human herpesvirus, type 8; a Papillomaviridae such as a Human papillomavirus; a Polyomaviridae such as a BK virus or a JC virus; a Poxviridae such as a Smallpox; a Hepadnaviridae such as a Hepatitis B virus; a Parvoviridae such as a Human bocavirus or a Parvovirus; a Astroviridae such as a Human astrovirus; a Caliciviridae such as a Norwalk virus; a Picornaviridae such as a coxsackievirus, a hepatitis A virus, a poliovirus, a rhinovirus; a Coronaviridae such as a Severe acute respiratory syndrome virus or a Wuhan coronavirus; a Flaviviridae such as a Hepatitis C virus, a yellow fever virus, a dengue virus, a West Nile virus; a Togaviridae such as a Rubella virus; a Hepeviridac such as a Hepatitis E virus; a Retroviridae such as a Human immunodeficiency virus (HIV); a Orthomyxoviridae such as an Influenza virus; a Arenaviridae such as a Guanarito virus, a Junin virus, a Lassa virus, a Machupo virus, a Sabiá virus; a Bunyaviridae such as a Crimean-Congo hemorrhagic fever virus; a Filoviridae such as a Ebola virus, a Marburg virus; a Paramyxoviridae such as a Measles virus, a Mumps virus, a Parainfluenza virus, a Respiratory syncytial virus, a Human metapneumovirus, a Hendra virus, a Nipah virus; a Rhabdoviridac such as a Rabies virus; a Hepatitis D virus; or a Reoviridae such as a Rotavirus, a Orbivirus, a Coltivirus, a Banna virus pathogen. In some embodiments, the microbial pathogen comprises a fungal pathogen. In some embodiments, the fungal pathogen comprises actinomycosis, allergic bronchopulmonary aspergillosis, aspergilloma, aspergillosis, athlete's foot, basidiobolomycosis, Basidiobolus ranarum, black piedra, blastomycosis, Candida krusei, candidiasis, chronic pulmonary aspergillosis, chrysosporium, chytridiomycosis, coccidioidomycosis, conidiobolomycosis, cryptococcosis, Cryptococcus gattii, deep dermatophytosis, dermatophyte, dermatophytid, dermatophytosis, endothrix, entomopathogenic fungus, epizootic lymphangitis, esophageal candidiasis, exothrix, fungal meningitis, fungemia, Geotrichum, Geotrichum candidum, histoplasmosis, lobomycosis, Massospora cicadina, Microsporum gypseum, muscardine, mycosis, myringomycosis, Neozygites remaudierei, Neozygites slavi, Ochroconis gallopava, Ophiocordyceps arborescens, Ophiocordyceps coenomyia, Ophiocordyceps macroacicularis, Ophiocordyceps nutans, oral candidiasis, paracoccidioidomycosis, pathogenic dimorphic fungi, penicilliosis, piedra, piedraia, Pneumocystis pneumonia, pseudallescheriasis, scedosporiosis, sporotrichosis, Tinea, Tinea barbae, Tinea capitis, Tinea corporis, Tinea cruris, Tinea faciei, Tinea incognito, Tinea nigra, Tinea pedis, Tinea versicolor, vomocytosis, white nose syndrome, zeaspora, or zygomycosis. In some cases, methods herein result in enrichment of a protozoon nucleic acid. In some cases, methods herein result in enrichment of a cancer nucleic acid. In some cases, methods herein result in enrichment of a fetal nucleic acid.

### Use of Endonuclease/Exonuclease Combinations in Targeted Depletion

The method described herein for depleting a first nucleic acid may result in a sequencing library with dramatically reduced complexity. Unwanted sequences are removed and the remaining sequences can be more readily analyzed by NGS techniques. The reduced complexity of the library can reduce the sequencer capacity required for clinical depth sequencing and/or reduce the computational requirement for accurate mapping of non-repetitive sequences. The sequence that is enriched can be searched in a bioinformatics database such as BLAST to determine the identity of the genes. The sequence information of the enriched nucleic acid can be used to determine the type of pathogen.

Through methods disclosed herein, a sample is treated so as to acquire exonuclease-protected ends, and then specific nucleic acids are cleaved so as to expose exonuclease-sensitive ends, such that a concurrent or subsequent exonuclease treatment selectively degrades nucleic acid cleavage products while leaving uncleaved, capped nucleic acids intact. Remaining nucleic acids are then used to prepare a sequencing library or otherwise assayed.

A number of workflows are consistent with the disclosure herein. Representative workflows are as follows, although variants are also contemplated.

Step 1: Nucleic Acid Extraction/Purification. A number of purification methods are consistent with the disclosure herein. In some cases, heat alone can rupture the cells. Sample sources may include saliva, blood, urine, CSF, skin, tissue, bone, etc. Each sample type and pathogen type may require different extraction and purification methods. Sample preparation approaches yielding nucleic acids suitable for downstream applications, such as genomic nucleic acids, circulating free nucleic acids, RNA or cDNA are consistent with the disclosure herein.

Step 2: DNA protection. Protecting the ends of DNA molecules from degradation can be achieved by ligating hairpin adapters, by ligating adapters using base modifications such as phosphorthioate, 2-O methyl, inverted dT or ddT, phosphorylation, C3 spacers, or simple modification to the ends of the sample nucleic acids without ligation of adapters. Tagmentation approaches of hairpin adapters or protected adapters may also be used.

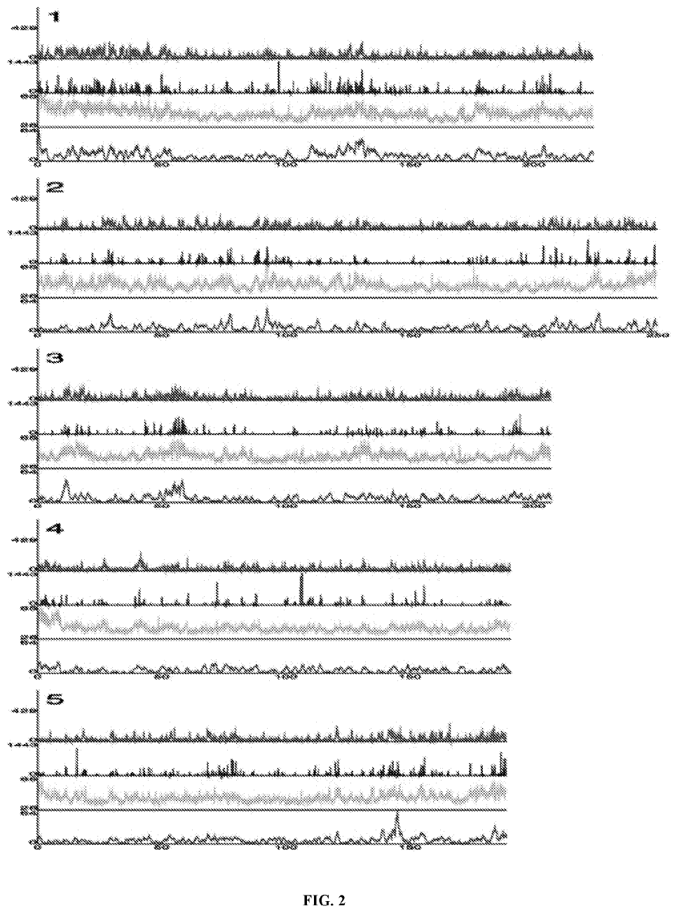

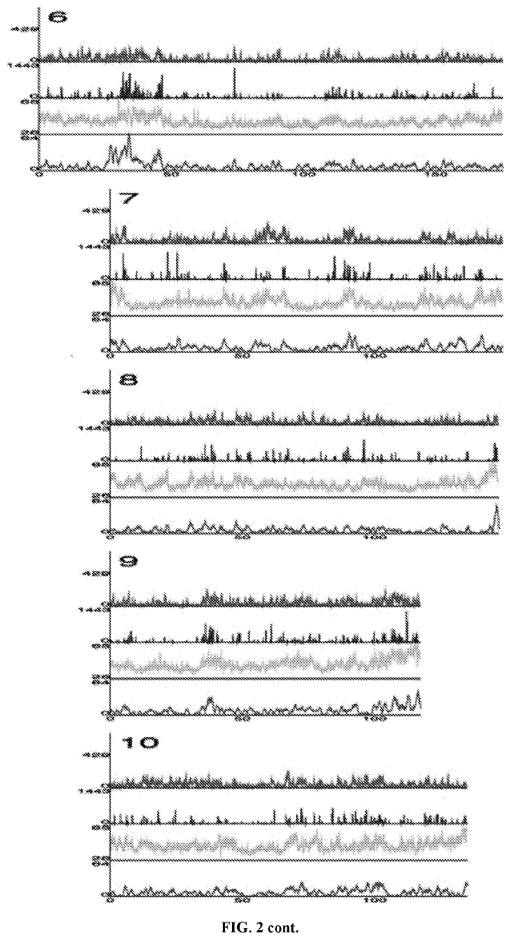

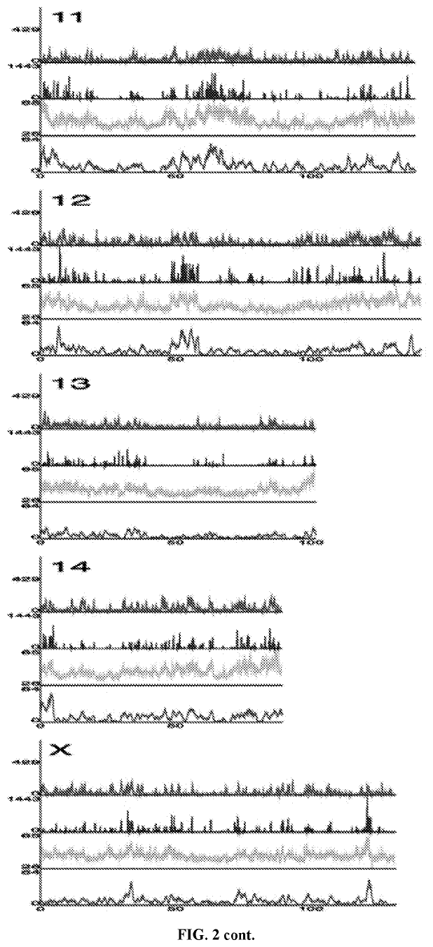

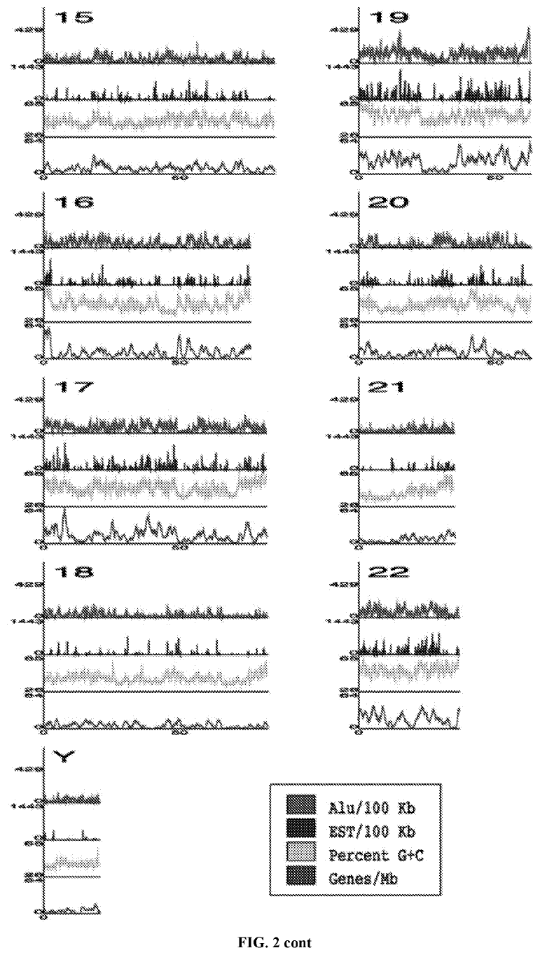

Step 3: endonuclease digestion of host molecules. This may be achieved with Restriction enzymes specific to host sequence motifs. This may include RNA guided endonuclease such as CRISPR systems or CRISPR derivatives. Examples of human specific sequence motifs may include Alu sequences. Alus are primate specific, are abundant in the human genome (over 1 M) and spaced throughput the genome. Examples of Alu specific restriction enzymes may include AluI, AsuHPI, Bpu10I, BssECI, BstDEI, BstMAI, HinfI, and BstTUI. FIG. 2 shows a map of Alu sequences in the human genome. Table 1 depicts the amount of Alu repeats which contain restriction enzymes recognition site at the certain positions. In some cases, an example of a human Alu monomer is 153 base pairs long, derived from 7SL RNA and having a sequence of

GCCGGGCGCGGTGGCGCGTGCCTGTAGTCCCagctACTCGGGAGGCTGA
GGCTGGAGGATCGCTTGAGTCCAGGAGTTCTGGGCTGTAGTGCGCTATG
CCGATCGGAATAGCCACTGCACTCCAGCCTGGGCAACATAGCGAGACCC
CGTCTC.

The recognition sequence of the Alu I endonuclease is 5′ ag/ct 3′; that is, the enzyme cuts the DNA segment between the guanine and cytosine residues (in lowercase above) PAM sequences for CRISPR-Cas9 shown above (underline).

TABLE 1
Amount of Alu repeats, which contain restriction enzymes recognition site at the certain positions

| | | Number of | % of | | | | | | |
|---|---|---|---|---|---|---|---|---|---|
| | | Alu | Alu | | % of Alu repeats with RE site in the main subfamilies | | | | |
| Restriction | | repeats | repeats | | | | | AluSg | |
| enzyme | | with | with a | AluJb | AluJo | AluSq | AluSx | (82, | AluY |
| (Recognition | Position | recognition | RE | (128,921 | (143,179 | (95,474 | (342,315 | 849 | (139,479 |
| site) | of site* | site* | site | copies) | copies) | copies) | copies) | copies) | copies) |
| AluI | 136 | 284,829 | 24 | 22 | 20 | 31 | 28 | 32 | 18 |
| (AGCT) | 168 | 751,006 | 63 | 53 | 47 | 76 | 72 | 78 | 78 |
| | 216 | 134,112 | 11 | 6 | 8 | 2 | 3 | 2 | 65 |
| | 228 | 261,288 | 22 | 20 | 33 | 22 | 23 | 22 | 19 |
| AsuHPI | 61 | 213,081 | 18 | 2 | 1 | 54 | 33 | 1 | 1 |
| (GGTGA) | 99/101 | 453,607 | 38 | 18 | 2 | 58 | 51 | 53 | 64 |
| | 259 | 243,227 | 20 | 23 | 18 | 11 | 27 | 28 | 20 |
| BPU10I | 46 | 229,143 | 19 | 18 | 19 | 21 | 22 | 22 | 16 |
| (CCTNAGC) | 181 | 633,313 | 53 | 41 | 35 | 63 | 59 | 68 | 72 |
| BssECI | 47 | 480,634 | 40 | 27 | 24 | 48 | 47 | 50 | 59 |
| (CCNNGG) | 89 | 190,713 | 16 | 47 | 46 | 3 | 6 | 2 | 2 |
| | 205 | 639,461 | 54 | 44 | 38 | 62 | 59 | 65 | 71 |
| | 255 | 607,377 | 51 | 42 | 35 | 62 | 59 | 64 | 72 |
| BstDEI | 47 | 273,350 | 23 | 23 | 25 | 24 | 26 | 25 | 18 |
| (CTNAG) | 65 | 281,670 | 24 | 16 | 8 | 60 | 41 | 1 | 1 |
| | 173** | 276,082 | 23 | 24 | 23 | 25 | 26 | 36 | 20 |
| | 182** | 760,759 | 64 | 55 | 49 | 75 | 72 | 78 | 79 |
| | 230 | 184,220 | 15 | 9 | 3 | 22 | 23 | 22 | 14 |
| BstMAI | 79/81 | 513,948 | 43 | 31 | 29 | 49 | 47 | 52 | 66 |
| (GTCTC) | 110/112 | 461,551 | 39 | 27 | 26 | 44 | 42 | 49 | 60 |
| | 270*** | 391,589 | 33 | 31 | 23 | 10 | 41 | 44 | 53 |
| | 278*** | 410,275 | 34 | 36 | 29 | 31 | 37 | 36 | 50 |
| Hinff | 193 | 344,902 | 29 | 6 | 4 | 53 | 46 | 50 | 5 |
| (GANTC) | 272 | 422,725 | 35 | 7 | 7 | 10 | 53 | 63 | 71 |
| Bst2UI | 3 | 257,322 | 22 | 20 | 19 | 25 | 26 | 25 | 20 |
| (CCWGG) | 71 | 183,209 | 15 | 37 | 43 | 3 | 10 | 2 | 2 |
| | 87/89 | 658,863 | 55 | 49 | 48 | 62 | 66 | 58 | 76 |
| | 138 | 270,331 | 23 | 23 | 21 | 2 | 28 | 27 | 23 |
| | 205 | 494,290 | 41 | 41 | 37 | 48 | 48 | 48 | 38 |
| | 255 | 640,134 | 54 | 42 | 36 | 62 | 60 | 65 | 72 |

Data are given for all analyzed sequences and for six main subfamilies
* - Positions and numbers of sites which present in more than 30% of Alu repeats are shown in bold.
** - Because of possible overlapping the following regions were considered. 168-177 and 178-187.
*** - Because of possible overlapping the following regions were considered: 265-274 and 275-283

Step 4: Library preparation. Once the host nucleic acids are removed, standard library preparation of the non-host molecules is performed for sequencing.

FIG. 1 illustrates a streamlined workflow of the host depletion method. Tagmentation procedures with protected adapter or hairpin sequences may be used to protect DNA molecules from exonuclease digestion. The ratio of Tn5 transposase to input DNA is optimized to produce protected molecules of sufficient length for sequencing. In the case of nanopore sequencing, the ideal molecule length would be greater than 1 kb. Following tagmentation, the endonuclease digestion is performed, followed immediately by exonuclease digestion of the cleaved molecules. The remaining molecules may be input directly into nanopore sequencing.

Alternatively, total nucleic acid (RNA and DNA) may be obtained. RNA is first converted into double stranded cDNA. Both ds cDNA and genomic DNA molecules are protected by means previously described. cDNA and DNA molecules are subjected to endonuclease digestion as previously described for host specific sequence motifs. Exposed ends of the unprotected endonuclease digested host molecules are degraded via exonuclease digestion.

The remaining non-host molecules are converted into sequencing libraries, sequenced and the data is analyzed to determine the pathogen present in the sample.

Methods described herein can include performing a genetic analysis of the second nucleic acid (e.g., enriched nucleic acid). Genome sequence databases can be searched to find sequences which are related to the second nucleic acid. The search can generally be performed by using computer-implemented search algorithms to compare the query sequences with sequence information stored in a plurality of databases accessible via a communication network, for example, the Internet. Examples of such algorithms include the Basic Local Alignment Search Tool (BLAST) algorithm, the PSI-blast algorithm, the Smith-Waterman algorithm, the Hidden Markov Model (HMM) algorithm, and other like algorithms.

### Definitions

A partial list of relevant definitions is as follows.

As used herein, the term “enriched” is used in a relative sense, such that a second nucleotide or population comprising a second nucleotide is enriched upon the selective depletion of a first nucleotide or population comprising a first nucleotide. It does not need increase in an absolute sense to be enriched. Rather, an absolute increase or a relative increase resulting from depletion or deletion of other nucleic acids may constitute ‘enrichment’ as used herein.

As used herein, the term “deplete” or “depleting” is used in a relative sense, such that a first nucleotide or population comprising a first nucleotide is degraded upon the selective preservation of a second nucleotide or population comprising a second nucleotide. It does not need decrease in an absolute sense to be depleted. Rather, an absolute decrease or a relative decrease resulting from preservation of other nucleic acids may constitute ‘depleting’ as used herein.

As used herein, “about” a given value is defined as +/−10% of said given value.

As used herein, NGS or Next Generation Sequencing may refer to any number of nucleic acid sequencing technologies, such as 5.1 Massively parallel signature sequencing (MPSS), Polony sequencing, 454 pyrosequencing, Illumina (Solexa) sequencing, SOLiD sequencing, Ion Torrent semiconductor sequencing, DNA nanoball sequencing, Heliscope single molecule sequencing, Single molecule real time (SMRT) sequencing, Tunnelling currents DNA sequencing, Sequencing by hybridization, Sequencing with mass spectrometry, Microfluidic Sanger sequencing, Microscopy-based techniques, RNAP sequencing, and In vitro virus high-throughput sequencing.

As used herein, to ‘modify’ a nucleic acid is to cause a change to a covalent bond in the nucleic acid, such as methylation, base removal, or cleavage of a phosphodiester backbone.

As used herein, to ‘direct transcription’ is to provide template sequence from which a specified RNA molecule can be transcribed.

“Amplified nucleic acid” or “amplified polynucleotide” includes any nucleic acid or polynucleotide molecule whose amount has been increased by any nucleic acid amplification or replication method performed in vitro as compared to its starting amount. For example, an amplified nucleic acid is optionally obtained from a polymerase chain reaction (PCR) which can, in some instances, amplify DNA in an exponential manner (for example, amplification to 2ⁿ copies in n cycles) wherein most products are generated from intermediate templates rather than directly from the sample template. Amplified nucleic acid is alternatively obtained from a linear amplification, where the amount increases linearly over time and which, in some cases, produces products that are synthesized directly from the sample.

The term “biological sample” or “sample” generally refers to a sample or part isolated from a biological entity. The biological sample, in some cases, shows the nature of the whole biological entity and examples include, without limitation, bodily fluids, dissociated tumor specimens, cultured cells, and any combination thereof. Biological samples come from one or more individuals. One or more biological samples come from the same individual. In one non limiting example, a first sample is obtained from an individual's blood and a second sample is obtained from an individual's tumor biopsy. Examples of biological samples include but are not limited to, blood, serum, plasma, nasal swab or nasopharyngeal wash, saliva, urine, gastric fluid, spinal fluid, tears, stool, mucus, sweat, earwax, oil, glandular secretion, cerebral spinal fluid, tissue, semen, vaginal fluid, interstitial fluids, including interstitial fluids derived from tumor tissue, ocular fluids, spinal fluid, throat swab, breath, hair, finger nails, skin, biopsy, placental fluid, amniotic fluid, cord blood, emphatic fluids, cavity fluids, sputum, pus, microbiota, meconium, breast milk and/or other excretions. In some cases, a blood sample comprises circulating tumor cells or cell free DNA, such as tumor DNA or fetal DNA. The samples include nasopharyngeal wash. Examples of tissue samples of the subject include but are not limited to, connective tissue, muscle tissue, nervous tissue, epithelial tissue, cartilage, cancerous or tumor sample, or bone. Samples are obtained from a human or an animal. Samples are obtained from a mammal, including vertebrates, such as murines, simians, humans, farm animals, sport animals, or pets. Samples are obtained from a living or dead subject. Samples are obtained fresh from a subject or have undergone some form of pre-processing, storage, or transport.

Nucleic acid sample as used herein refers to a nucleic acid sample for which the first nucleic acid is to be determined, A nucleic acid sample is extracted from a biological sample above, in some cases. Alternatively, a nucleic acid sample is artificially synthesized, synthetic, or de novo synthesized in some cases. The DNA sample is genomic in some cases, while in alternate cases the DNA sample is derived from a reverse-transcribed RNA sample.

“Bodily fluid” generally describes a fluid or secretion originating from the body of a subject. In some instances, bodily fluid is a mixture of more than one type of bodily fluid mixed together. Some non-limiting examples of bodily fluids include but are not limited to: blood, urine, bone marrow, spinal fluid, pleural fluid, lymphatic fluid, amniotic fluid, ascites, sputum, or a combination thereof.

“Complementary” or “complementarity,” or, in some cases more accurately “reverse-complementarity” refer to nucleic acid molecules that are related by base-pairing. Complementary nucleotides are, generally, A and T (or A and U), or C and G (or G and U). Functionally, two single stranded RNA or DNA molecules are complementary when they form a double-stranded molecule through hydrogen-bond mediated base paring. Two single stranded RNA or DNA molecules are said to be substantially complementary when the nucleotides of one strand, optimally aligned and with appropriate nucleotide insertions or deletions, pair with at least about 90% to about 95% or greater complementarity, and more preferably from about 98% to about 100%) complementarity, and even more preferably with 100% complementarity. Alternatively, substantial complementarity exists when an RNA or DNA strand will hybridize under selective hybridization conditions to its complement. Selective hybridization conditions include, but are not limited to, stringent hybridization conditions and not stringent hybridization conditions. Hybridization temperatures are generally at least about 2° C. to about 6° C. lower than melting temperatures (Tₘ).

“Double-stranded” refers, in some cases, to two polynucleotide strands that have annealed through complementary base-pairing, such as in a reverse-complementary orientation.

“Known oligonucleotide sequence” or “known oligonucleotide” or “known sequence” refers to a polynucleotide sequence that is known. In some cases, a known oligonucleotide sequence corresponds to an oligonucleotide that has been designed, e.g., a universal primer for next generation sequencing platforms (e.g., Illumina, 454), a probe, an adaptor, a tag, a primer, a molecular barcode sequence, an identifier. A known sequence optionally comprises part of a primer. A known oligonucleotide sequence, in some cases, is not actually known by a particular user but is constructively known, for example, by being stored as data accessible by a computer. A known sequence is optionally a trade secret that is actually unknown or a secret to one or more users but is known by the entity who has designed a particular component of the experiment, kit, apparatus or software that the user is using.

“Library” in some cases refers to a collection of nucleic acids. A library optionally contains one or more target fragments. In some instances the target fragments comprise amplified nucleic acids. In other instances, the target fragments comprise nucleic acid that is not amplified. A library optionally contains nucleic acid that has one or more known oligonucleotide sequence(s) added to the 3′ end, the 5′ end or both the 3′ and 5′ end. The library is optionally prepared so that the fragments contain a known oligonucleotide sequence that identifies the source of the library (e.g., a molecular identification barcode identifying a patient or DNA source). In some instances, two or more libraries are pooled to create a library pool. Libraries are optionally generated with other kits and techniques such as transposon mediated labeling, or “tagmentation” as known in the art. Kits are commercially available. One non-limiting example of a kit is the Illumina NEXTERA kit (Illumina, San Diego, Calif.).

The term “polynucleotides” or “nucleic acids” includes but is not limited to various DNA, RNA molecules, derivatives or combination thereof. These include species such as dNTPs, ddNTPs, DNA, RNA, peptide nucleic acids, cDNA, dsDNA, ssDNA, plasmid DNA, cosmid DNA, chromosomal DNA, genomic DNA, viral DNA, bacterial DNA, mtDNA (mitochondrial DNA), mRNA, rRNA, tRNA, nRNA, siRNA, snRNA, snoRNA, scaRNA, microRNA, dsRNA, ribozyme, riboswitch and viral RNA.

Before the present methods, compositions and kits are described in greater detail, it is to be understood that this invention is not limited to particular method, composition or kit described, as such may, of course, vary. It is also to be understood that the terminology used herein is for the purpose of describing particular embodiments only, and is not intended to be limiting, since the scope of the present invention will be limited only by the appended claims as construed herein. Examples are put forth so as to provide those of ordinary skill in the art with a more complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein are optionally used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method is contemplated to be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Detection of a Pathogen in an Infectious Disease Outbreak

A population of subjects present at a clinic with similar symptoms and none of them test positive for a known pathogen. Blood samples are obtained from each subject and nucleic acids are extracted from the samples. Protected oxford nanopore adapters are ligated onto 5′ and 3′ ends of the sample nucleic acids which contain subject or host nucleic acids and pathogen nucleic acids. The host nucleic acids are targeted using a CRISPR/Cas targeted for the subject nucleic acids to create double stranded breaks in the host nucleic acids. An exonuclease is added to the samples. The exonuclease cannot digest the modified adapters so only nucleic acids that have double stranded breaks are digested by the exonuclease. The remaining nucleic acids are purified and sequenced using a nanopore sequencer in order to identify the common pathogen.

### Example 2: A Comparison of Ribodepletion of Different Size E. coli rRNA Libraries with E. coli-Specific and Pan-Bacterial CRISPR Guides

Ribodepletion was performed at 37° C. for 1 hour with a target site:Cas9:sgRNA ratio of 1:2000:5000. Two types of NEBNext Ultra II RNA libraries were prepared: 1) a large fragment library (5 min fragmentation & “520 bp” dual bead size selection (not typical of most RNA libraries produced); and 2) a small fragment library (15 min fragmentation & single 1× bead size selection (more akin to typical RNA libraries).

Ribodepletion was performed in one of three ways: 1) 1 ng input, 1× Ampure bead cleanup, gel size selection (low input and higher duplication rate; probably not ideal for multiplexing; most stringent size selection (involving gels)); 2) 10 ng input, 0.6× Ampure bead cleanup (higher input, moderate size selection); and 3) 10 ng input, 1× Ampure bead cleanup (higher input, weaker size selection).

Ribodepletion with E. coli-specific guides resulted in highest (>99%) under optimal conditions (large fragment library, stringent size selection). It was lower with smaller libraries: with 0.6× final bead size selection ribodepletion was ˜95%, while ribodepletion was 85-90% with 1× final bead size selection).

Ribodepletion with pan-bacterial guides was also highest (78-90%) with large fragment libraries, low input and gel-based size selection. Ribodepletion with pan-bacterial guides is substantially lower (˜50%) with small fragment libraries, higher library input and 0.6× Ampure bead cleanup.

Ribodepletion results for each library is described in Table 2 below.

TABLE 2
Ribodepletion Results

| Sample | Ribodepletion |
|---|---|
| Mock-520_S2_L001 | N/A |
| A1-Ecoli-520-PB_S6_L001 | 99.75 |
| A2-Ecoli-520-PB_S21_L001 | 99.70 |
| A1-Ag-520-PB_S15_L001 | 79.94 |
| A2-Ag-520-PB_S10_L001 | 88.78 |
| Mock-ss-06x-A_S17_L001 | N/A |
| A1-Ecoli-ss-PB_S11_L001 | 96.04 |
| A2-Ecoli-ss-PB_S18_L001 | 94.45 |
| A1-Ag-ss-PB_S12_L001 | 49.59 |
| A2-Ag-ss-PB_S3_L001 | 52.56 |
| Mock-ss-1x-B_S14_L001 | N/A |
| B1-Ecoli-ss-PB_S7_L001 | 86.92 |
| B2-Ecoli-ss-PB_S22_L001 | 88.14 |
| B1-Ag-ss-PB_S5_L001 | 26.15 |
| B2-Ag-ss-PB_S23_L001 | 25.03 |

### Example 3: Directional RNA Library Prep Libraries from E. coli Total RNA

NEBNext Ultra II Directional RNA Library Prep was used to prepare libraries from 100 ng of E. coli total RNA.

CRISPR guides were designed to cover all bacterial species. The DNA oligonucleotides containing the sequences of the 12,368 guides were produced by Agilent on an array.

The oligos were amplified by PCR, then transcribed using a 5′ T7 promoter sequence by T7 RNA polymerase-mediated in vitro transcription (IVT) using each of three IVT kits (Agilent SureGuide T7, Thermo Fisher MegaScript T7 and Lucigen AmpliScribe T7-Flash).

1 ng of the NEB library was treated with Cas9 and sgRNA at a target site:Cas9:sgRNA ratio of 1:2000:5000.

Ribodepletion was followed by 0.6× Ampure bead size selection, PCR (15 and 11 cycles for CRISPR-treated and untreated samples, respectively) and a 1× Ampure bead size selection.

The ribodepleted libraries were run on a gel and 500-900 bp fragments were gel purified and loaded on a MiSeq instrument.

CRISPR guides specific to S. aureus (˜100 custom made guides) were the most effective in depleting the samples of ribosomal RNA (0.05% and 0.11% of reads aligning to 16S and 23S rRNA respectively). Percentage ribodepletion was greater than 99.5%.

Percentage ribodepletion was highest with the Agilent IVT produced CRISPR pan-bacterial guides (94-96% rRNA removed).

The Lucigen IVT kit was less effective in rRNA removal with % ribodepletion rates of 91-94%. The ThermoFisher IVT kit was least effective in rRNA removal with ribodepletion rates of 79-92%.

Ribodepletion for each library is summarized in Table 3 below.

TABLE 3
Ribodepletion Results

| Sample | % ribodepletion |
|---|---|
| PB3-Sa-Mock-untreat_S10_L001 | N/A |
| PB3-Sa-Ag50-A1_S1_L001 | 95.9344629 |
| PB3-Sa-Ag100-A2_S2_L001 | 96.12857488 |
| PB3-Sa-Ag200-A3_S3_L001 | 94.43268946 |
| PB3-Sa-L100-L1_S7_L001 | 93.87941026 |
| PB3-Sa-L500-L2_S8_L001 | 94.87793448 |
| PB3-Sa-TF50-TF1_S4_L001 | 91.75859767 |
| PB3-Sa-TF50-2x-TF3_S6_L001 | 79.68662441 |
| PB3-Sa-TF100-TF2_S5_L001 | 88.86264803 |
| PB3-Sa-PC-dr_S9_L001 | 99.82271034 |

### Example 4: Human Ribosomal RNA Depletion

Total RNA was obtained from brain, kidney, liver, and heart. A NGS library was prepared from the total RNA. CRISPR Cas9 was used to digest the ribosomal RNA in the NGS library. A size selection was performed using Ampure beads and PCR was performed on the size selected library.

Library characteristics are summarized in Table 4 below.

TABLE 4
Library Characteristics

| | Homo sapiens | Homo sapiens |
|---|---|---|
| | mock treated: no | treated with |
| | Cas9, no gRNA | Cas9 + gRNA |
| Genome alignment rate | 99.3% | 99.3% |
| Duplication rate (2 million reads) | 52.5% | 4.0% |
| Alignment rate (18 S rRNA) | 11.4% | 0.0% |
| Alignment rate (28 S rRNA) | 22.7% | 0.1% |
| Alignment rate (12 S rRNA) | 1.2% | 0.0% |
| Alignment rate (16 S rRNA) | 1.4% | 0.0% |
| Alignment rate (exon) | 3.7% | 48.5% |
| Alignment rate (intron) | 3.2% | 29.6% |
| Alignment rate (intragenic) | 15.9% | 13.6% |
| rRNA depletion rate | N/A | 99.0% |

Ribosomal depletion data for each sample is summarized in Table 5 below.

TABLE 5
rRNA Depletion Data from Multiplexed Libraries

| | Genome | 12S | 16S | 18S | 28S | | | |
|---|---|---|---|---|---|---|---|---|
| | alignment | rRNA | rRNA | rRNA | rRNA | CDS | UTR | Intron |
| | rate | alignment | alignment | alignment | alignment | alignment | alignment | alignment |
| Brain (1) | 99.63% | 0.00% | 0.01% | 0.04% | 0.13% | 24.07% | 21.96% | 30.93% |
| Brain (2) | 99.66% | 0.00% | 0.01% | 0.04% | 0.13% | 25.90% | 22.15% | 29.66% |
| Kidney | 99.60% | 0.00% | 0.00% | 0.03% | 0.10% | 27.75% | 18.55% | 29.42% |
| (1) | | | | | | | | |
| Kidney | 99.63% | 0.00% | 0.00% | 0.03% | 0.09% | 28.20% | 18.61% | 28.90% |
| (2) | | | | | | | | |
| Liver (1) | 99.68% | 0.00% | 0.00% | 0.04% | 0.11% | 46.72% | 17.25% | 15.88% |
| Liver (2) | 99.71% | 0.00% | 0.00% | 0.04% | 0.15% | 46.26% | 17.28% | 16.03% |
| Heart (1) | 99.59% | 0.00% | 0.01% | 0.03% | 0.10% | 34.05% | 16.81% | 11.25% |
| Heart (2) | 99.66% | 0.01% | 0.01% | 0.05% | 0.15% | 34.15% | 17.41% | 11.45% |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

What is claimed is:

1. A method of depleting a first nucleic acid from a sample, comprising
(a) providing a sample comprising the first nucleic acid and a second nucleic acid;
(b) capping 5′ and 3′ ends of the first nucleic acid and the second nucleic acid;
(c) contacting the sample to an endonuclease to form at least one cleaved first nucleic acid, wherein the endonuclease cleaves the first nucleic acid but does not cleave the second nucleic acid; and
(d) contacting the sample to an exonuclease.

2. The method of claim 1, wherein capping comprises modifying the 5′ or 3′ ends of the first and second nucleic acids to make the first and the second nucleic acids resistant to exonuclease degradation.

3. The method of claim 1, wherein capping comprises attaching adaptors to the 5′ and 3′ ends of the first nucleic acid and the second nucleic acid.

4. The method of claim 3, wherein the adaptor is a hairpin or a linear adaptor.

5. The method of claim 4, wherein the linear adaptor is selected from the group consisting of phosphorthioate, 2-O methyl, inverted dT, inverted ddT, phosphorylation, and C3 spacers.

6. The method of claim 1, wherein the endonuclease is a restriction enzyme specific to at least one site on the first nucleic acid.

7. The method of claim 1, wherein the endonuclease comprises at least one selected from Clustered Regulatory Interspaced Short Palindromic Repeat (CRISPR)/Cas system protein-guide RNA (gRNA) complexes, Zinc Finger Nucleases (ZFN), and Transcription activator like effector nucleases.

8. The method of claim 7, wherein the gRNAs are complementary to at least one site on the first nucleic acid to generate cleaved first nucleic acids capped only on one end.

9. The method of claim 1, wherein the endonuclease comprises an Alu specific restriction enzyme.

10. The method of claim 1, wherein the first nucleic acid comprises at least one sequence that maps to at least one nucleic acid selected from the group consisting of AluI, AsuHPI, Bpu10I, BssECI, BstDEI, BstMAI, HinfI, and BstTUI.

11. The method of claim 1, wherein the cleaved first nucleic acid is capped at only one end.

12. The method of claim 3, wherein the cleaved first nucleic acid has a first end that is attached to an adaptor and a second end that is not attached to an adaptor.

13. The method of claim 1, comprising extracting the first and second nucleic acids from the sample and purifying the first and second nucleic acids.

14. The method of claim 1, wherein the first and second nucleic acids comprise any one of single stranded DNA, double stranded DNA, single stranded RNA, double stranded RNA, cDNA, synthetic DNA, artificial DNA, and DNA/RNA hybrids.

15. The method of claim 1, comprising amplifying the second nucleic acid.

16. The method of claim 1, comprising sequencing the second nucleic acid.

17. The method of claim 1, comprising sequencing the second nucleic acid through a second-generation sequencing method.

18. The method of claim 1, comprising sequencing the second nucleic acid through a nanopore sequencing method.

19. The method of claim 1, wherein the first nucleic acid comprises a nucleic acid from a human.

20. The method of claim 1, wherein the first nucleic acid comprises a host nucleic acid.

21. The method of claim 1, wherein the first nucleic acid comprises a repetitive nucleic acid.

22. The method of claim 1, wherein the first nucleic acid comprises a centromere nucleic acid.

23. The method of claim 1, wherein the first nucleic acid comprises a transposon.

24. The method of claim 1, wherein the first nucleic acid comprises an Alu element.

25. The method of claim 1, wherein the second nucleic acid comprises a microbiome nucleic acid.

26. The method of claim 1, wherein the second nucleic acid comprises an oncogenic nucleic acid.

27. The method of claim 1, wherein the second nucleic acid comprises a symbiont nucleic acid.

28. The method of claim 1, wherein the second nucleic acid comprises a single-copy region of a haploid genome.

29. The method of claim 1, wherein the second nucleic acid comprises a nucleic acid from a pathogen.

30. The method of claim 29, wherein the pathogen is selected from the group consisting of a virus, a bacterium, a fungus, and a protozoon.

31. The method of claim 29, comprising sequencing the second nucleic acid and determining the type of the pathogen.

32. The method of claim 1, wherein the second nucleic acid comprises a nucleic acid from a tumor.

33. The method of claim 1, wherein the sample wherein the sample is selected from saliva, blood, plasma, serum, mucous, feces, urine, cerebrospinal fluid (CSF), skin, tissue, and bone.

34. A composition comprising a mixture of a first nucleic acid and a second nucleic acid, wherein the first nucleic acid and the second nucleic acid are capped at 3′ and 5′ ends, and wherein the first nucleic acid is complexed to an endonuclease and the second nucleic acid is not complexed to the endonuclease.

35. The composition of claim 34, wherein the endonuclease comprises at least one selected from Clustered Regulatory Interspaced Short Palindromic Repeat (CRISPR)/Cas system protein-gRNA complex, Zinc Finger Nucleases (ZFN), and Transcription activator like effector nucleases.

36. The composition of claim 34, wherein endonuclease comprises a Clustered Regulatory Interspaced Short Palindromic Repeat (CRISPR)/Cas system protein-guide RNA (gRNA) complexes.

37. The composition of claim 36, wherein the gRNA is complementary to at least one site on the first nucleic acid to generate cleaved first nucleic acids capped only on one end.

38. The composition of claim 34, wherein the endonuclease comprises an Alu specific restriction enzyme.

39. The composition of claim 34, wherein the first nucleic acid comprises at least one sequence that maps to at least one nucleic acid selected from the group consisting of AluI, AsuHPI, Bpu10I, BssECI, BstDEI, BstMAI, HinfI, and BstTUI.

40. The composition of claim 34, wherein the first nucleic acid comprises a repetitive region.

41. The composition of claim 34, wherein the first nucleic acid comprises an Alu repeat.

42. The composition of claim 34, wherein the first nucleic acid comprises a nucleic acid from a human.

43. The composition of claim 34, wherein the second nucleic acid comprises a nucleic acid from a pathogen.

44. The composition of claim 43, wherein the pathogen is selected from the group consisting of a virus, bacterial, fungus, and protozoa.

45. The composition of claim 34, wherein the second nucleic acid comprises a nucleic acid from a tumor.

46. The composition of claim 34, wherein the first nucleic acid comprises a host nucleic acid.

47. The composition of claim 34, wherein the first nucleic acid comprises a repetitive nucleic acid.

48. The composition of claim 34, wherein the first nucleic acid comprises a centromere nucleic acid.

49. The composition of claim 34, wherein the second nucleic acid comprises a microbiome nucleic acid.

50. The composition of claim 34, wherein the second nucleic acid comprises an oncogenic nucleic acid.

51. The composition of claim 34, wherein the second nucleic acid comprises a symbiont nucleic acid.

52. The composition of claim 34, wherein the second nucleic acid comprises a single-copy region of a haploid genome.

53. The composition of claim 34, wherein the first nucleic acid comprises a transposon.

54. The composition of claim 34, wherein the first nucleic acid comprises an Alu element.
